SEQUENCE LISTING (34 sequences)

SEQ ID NO: 1 - DNA, 1200 bp, Homo sapiens
  misc_feature 1..1200: note = Description of sequence: receptor for Mu opioids
  source 1..1200: mol_type = genomic DNA
atggattcttcagcagctccaacaaatgcctctaactgcactgatgctttggcttattcttcttgttctccagctccatctccaggttcttgggttaatttgtctcatttggatggtaacttgtctgatccatgtggtccaaatagaactgacttaggtggtagagattctttgtgtccaccaactggttctccatctatgattactgctattaccattatggccctgtactctatcgtttgtgttgttggtttgttcggtaacttcttggttatgtacgttatcgtcagatacaccaagatgaagactgctaccaacatctacattttcaacttggctttggctgatgcattggcaacttctactttgccatttcaatccgtcaattacttgatgggtacttggccatttggtactattttgtgcaagatcgttatctccatcgactactacaacatgttcacctctattttcaccttgtgcaccatgtccgttgatagatatattgctgtttgccatccagttaaggccttggattttagaactccaagaaacgccaagattatcaacgtctgcaattggattttgtcctccgctattggtttgccagttatgtttatggctactaccaagtacagacaaggttctattgattgcactttgaccttctctcatccaacttggtattgggaaaacttgttgaagatctgcgttttcattttcgccttcatcatgccagttttgattatcactgtttgctacggcttgatgatcttgagattgaagtctgtcagaatgctgtccggttctaaagaaaaggatagaaacttgagaaggatcaccagaatggttttggttgttgttgctgttttcatcgtttgttggaccccaattcatatctacgttatcatcaaggctttggtcactattccagaaactactttccaaactgtttcctggcatttctgtattgctttgggttacactaactcttgtttgaacccagtcttgtacgcttttttggacgaaaacttcaagagatgcttccgtgaattctgtattccaacctcctctaacatcgaacaacaaaactctaccagaatcagacagaacactagagatcatccatctactgctaacactgttgatagaactaaccaccaattggagaacttggaagctgaaactgctccattgcca

SEQ ID NO: 2 - DNA, 1116 bp, Homo sapiens
  misc_feature 1..1116: note = Description of sequence: receptor for Mu opioids
  source 1..1116: mol_type = genomic DNA
atggaaccagctccttctgcaggtgctgaattgcagccacctttgtttgctaatgcttctgatgcttatccatctgcttttccatcagctggtgctaatgcatcaggtccaccaggtgctagatctgcttcttctttggctttggctattgcaattactgccttgtattctgctgtttgtgctgttggtttgttgggtaatgttttggttatgttcggtatcgtcagatacactaagatgaagactgctaccaacatctacattttcaatttggcattggctgatgctttggccacttctactttgccatttcaatctgctaagtacttgatggaaacttggccattcggtgaattattgtgtaaggccgttttgtccatcgactactacaatatgttcacctccattttcaccctgaccatgatgtctgttgataggtatattgcagtttgccatccagttaaggctttggattttagaactccagctaaggccaagttgattaacatctgtatttgggttttagcctccggtgttggtgttccaattatggttatggctgttactagaccaagagatggtgctgttgtatgtatgttgcaatttccttctccatcttggtactgggatactgttactaagatttgcgttttcttgttcgccttcgttgttccaatcttgattattaccgtttgctacggcttgatgttgttgagattgagatccgtcagattgctgtctggttctaaagaaaaggacagatccttgagaagaatcaccagaatggttttggttgttgttggtgctttcgttgtttgctgggctccaattcatattttcgttatcgtttggaccttggtcgacatcgatagaagagatccattagttgttgctgccttgcatttgtgtattgctttgggttatgctaactcctcattgaatccagtcttgtacgcttttttggacgaaaacttcaagagatgcttcagacaattgtgtagaaagccatgtggtagaccagatccatcttcattttctagagctagagaagctactgccagagaaagagttactgcttgtactccatctgatggtccaggtggtggtgctgcagct

SEQ ID NO: 3 - DNA, 1140 bp, Homo sapiens
  misc_feature 1..1140: note = Description of sequence: receptor for Mu opioids
  source 1..1140: mol_type = genomic DNA
atggattctccaattcaaattttcagaggtgaaccaggtccaacttgtgctccatctgcttgtttgccaccaaattcttcagcttggtttccaggttgggctgaaccagattctaatggttctgctggttctgaagatgctcaattggaaccagctcatatttctccagctattccagttattatcaccgccgtttactctgttgttttcgttgttggtttggtcggtaactctttggttatgttcgttatcatcaggtacaccaagatgaagactgctaccaacatctacattttcaacttggctttggctgatgctttggttactactactatgccattccaatctaccgtctacttgatgaattcttggccatttggtgatgtcttgtgcaagatcgttatctccattgactactacaacatgttcacctccattttcactctgaccatgatgtccgttgataggtatattgctgtttgccatccagttaaggctttggattttagaactccattgaaggccaagatcatcaacatctgtatttggctgttgtcctcctctgttggtatttctgctatagttttaggtggcaccaaggttagagaagatgttgacgttattgaatgctccttgcaattcccagatgatgattattcttggtgggacttgttcatgaagatctgcgttttcattttcgccttcgttatcccagttttgatcatcatcgtttgctacaccttgatgatcctgagattgaaatctgtcagactgttgtctggttccagagaaaaggatagaaacttgagaagaatcaccagattggttttggttgttgttgctgtttttgttgtttgctggaccccaattcacatcttcattttggttgaagctttgggttctacctctcattctactgctgctttgtcatcttactatttctgcattgccttgggttacaccaactcttcattgaatccaatcttgtacgctttcttggacgaaaacttcaagagatgcttcagagatttctgcttcccattgaagatgagaatggaaagacaatccacctccagagttagaaacactgttcaagatccagcttacttgagagatatcgatggtatgaacaagccagtc

SEQ ID NO: 4 - DNA, 126 bp, Homo sapiens
  misc_feature 1..126: note = Description of sequence: receptor for Mu opioids
  source 1..126: mol_type = genomic DNA
agcagcatggtaggatatagtacagcttctttagaaatttcaacgtatgctggcagtgccaacagcttactggccggtagtggtttaagtgtcttcattgcgtccttattgctggcaattatttaa

SEQ ID NO: 5 - DNA, 24 bp, Homo sapiens
  misc_feature 1..24: note = Description of sequence: receptor for Mu opioids
  source 1..24: mol_type = genomic DNA
tatgggggtttcctgaggcgtata

SEQ ID NO: 6 - DNA, 39 bp, Homo sapiens
  misc_feature 1..39: note = Description of sequence: receptor for Mu opioids
  source 1..39: mol_type = genomic DNA
tatgggggtttcctgaggcgtataaggccgaagttaaag

SEQ ID NO: 7 - DNA, 255 bp, Homo sapiens
  misc_feature 1..255: note = Description of sequence: receptor for Mu opioids
  source 1..255: mol_type = genomic DNA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggagtctctttggataaaaga

SEQ ID NO: 8 - DNA, 39 bp, Homo sapiens
  misc_feature 1..39: note = Description of sequence: receptor for Mu opioids
  source 1..39: mol_type = genomic DNA
tggcattggttgcaattgaaaccaggtcaaccaatgtat

SEQ ID NO: 9 - DNA, 54 bp, synthetic construct
  misc_feature 1..54: note = Description of sequence: Linker
  source 1..54: mol_type = other DNA
ggcggtggaagcggtggtggagggagcggtgggggttctgggggcggagggagt

SEQ ID NO: 10 - protein, 8 aa, synthetic construct
ADMSLRRI

SEQ ID NO: 11 - protein, 8 aa, synthetic construct
WWPDLRRI

SEQ ID NO: 12 - protein, 8 aa, synthetic construct
YGFDLRRI

SEQ ID NO: 13 - protein, 8 aa, synthetic construct
YGMELRRI

SEQ ID NO: 14 - protein, 8 aa, synthetic construct
YGGFGGSG

SEQ ID NO: 15 - protein, 8 aa, synthetic construct
YGGFGSGR

SEQ ID NO: 16 - protein, 8 aa, synthetic construct
YGGFSSGL

SEQ ID NO: 17 - protein, 8 aa, synthetic construct
YGGFSSRG

SEQ ID NO: 18 - protein, 8 aa, synthetic construct
YGGFSSSG

SEQ ID NO: 19 - protein, 8 aa, synthetic construct
  REGION 1..8: note = Description of sequence: HsKOR-specific peptide
CDIQLRRI

SEQ ID NO: 20 - protein, 8 aa, synthetic construct
  REGION 1..8: note = Description of sequence: HsKOR-specific peptide
SAWPLRRI

SEQ ID NO: 21 - protein, 8 aa, synthetic construct
  REGION 1..8: note = Description of sequence: HsKOR-specific peptide
SHYVLRRI

SEQ ID NO: 22 - protein, 8 aa, synthetic construct
  REGION 1..8: note = Description of sequence: HsKOR-specific peptide
SVPFLRRI

SEQ ID NO: 23 - protein, 8 aa, synthetic construct
  REGION 1..8: note = Description of sequence: HsKOR-specific peptide
YQRFLRRI

SEQ ID NO: 24 - protein, 8 aa, synthetic construct
  REGION 1..8: note = Description of sequence: HsKOR-specific peptide
YFFGYRLA

SEQ ID NO: 25 - protein, 8 aa, synthetic construct
  REGION 1..8: note = Description of sequence: HsKOR-specific peptide
YGGFYRLR

SEQ ID NO: 26 - protein, 8 aa, synthetic construct
  REGION 1..8: note = Description of sequence: HsKOR-specific peptide
YGGFYSRW

SEQ ID NO: 27 - protein, 8 aa, synthetic construct
  REGION 1..8: note = Description of sequence: HsKOR-specific peptide
YGGFYSYA

SEQ ID NO: 28 - protein, 8 aa, synthetic construct
  REGION 1..8: note = Description of sequence: HsMOR-specific peptide
GLVNLRRI

SEQ ID NO: 29 - protein, 8 aa, synthetic construct
  REGION 1..8: note = Description of sequence: HsMOR-specific peptide
LVLLLRRI

SEQ ID NO: 30 - protein, 8 aa, synthetic construct
  REGION 1..8: note = Description of sequence: HsMOR-specific peptide
YGGFMGSV

SEQ ID NO: 31 - protein, 8 aa, synthetic construct
  REGION 1..8: note = Description of sequence: HsMOR-specific peptide
YGGFMNYP

SEQ ID NO: 32 - protein, 8 aa, synthetic construct
  REGION 1..8: note = Description of sequence: HsMOR-specific peptide
YGGFMVAT

SEQ ID NO: 33 - protein, 8 aa, synthetic construct
  REGION 1..8: note = Description of sequence: HsMOR-specific peptide
YGGFRCCA

SEQ ID NO: 34 - protein, 18 aa, synthetic construct
  REGION 1..18: note = Description of sequence: Linker peptide
GGGSGGGGSGGGSGGGGS

(19) United States
(12) Patent Application Publication
(10) Pub. No.: US 2026/0308224 A1
(43) Pub. Date: Oct. 8, 2026

(54) METHODS AND COMPOSITIONS FOR DISPLAYING PEPTIDE LIBRARIES

(71) Applicant: BOARD OF REGENTS, THE UNIVERSITY OF TEXAS SYSTEM, Austin, TX (US)

(72) Inventors: Andrew ELLINGTON, Austin, TX (US); Colleen MULVIHILL, Austin, TX (US); Jimmy GOLLIHAR, Hewitt, TX (US); Elizabeth GARDNER, Houston, TX (US); Edward MARCOTTE, Austin, TX (US)

(21) Appl. No.: 19/144,201

(22) PCT Filed: Jan. 4, 2024
(86) PCT No.: PCT/US2024/010251

§ 371 Date: Jun. 27, 2025

Related U.S. Application Data
(60) Provisional application No. 63/478,406, filed on Jan. 4, 2023.

(51) Int. Cl. C07K 14/675; C07K 14/70; C12N 15/63; G01N 33/50; G01N 33/569

(52) U.S. Cl. CPC C07K 14/675; C07K 14/70; C12N 15/63; G01N 33/502; G01N 33/569; C07K 2319/036; G01N 2333/70; G01N 2500/04; G01N 2500/10

(57) ABSTRACT

A non-naturally occurring test molecule for use in a cell-based screening method, wherein the molecule comprises: a molecule of interest comprising a message/address peptide; a linker sequence; and an anchor sequence which anchors the test molecule to a plasma membrane of the cell. A method of screening to determine interaction between a capture protein and a molecule of interest, the method comprising the steps of: providing the test molecule; providing a eukaryotic cell, wherein said eukaryotic cell comprises a non-endogenous capture protein; allowing the molecule of interest and the non-endogenous capture protein to interact; and determining interaction between the molecule of interest and the capture protein.

### CROSS-REFERENCE TO RELATED APPLICATIONS

This application claims benefit of U.S. Provisional Application No. 63/478,406, filed Jan. 4, 2023, incorporated herein by reference in its entirety.

### FEDERALLY SPONSORED RESEARCH AND DEVELOPMENT

This invention was made with government support under Grant no. R35 GM122480 awarded by the National Institutes of Health and Grant no. W911NF-17-2-0091 awarded by the Army Research Office. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Dec. 19, 2023, is entitled “10046-513WO1_ST26.xml”, and is 43,383 bytes in size.

### BACKGROUND OF THE INVENTION

Cells utilize receptors to sense external stimuli like light, peptides and small molecules. These receptors then coordinate with machinery inside of the cell—often multiple signaling cascades—to create an output to that response. Morphological changes during mammalian development, yeast cell mating, and immune system response are just a few examples of processes dependent on receptors sensing external stimuli, then catalyzing essential cellular feedback.

G protein-coupled receptors (GPCRs) encompass the largest class of receptors in humans (Lengger et al. 2019). These receptors are characterized by seven membrane-spanning domains, with N-termini exposed outside of the cell, and C-termini at the cell cytoplasm (Zhou et al. 2019). It was found that 831 GPCRs respond to a variety of external stimuli and are important drug targets—roughly 34% of all FDA-approved drugs target a GPCR (Hauser et al. 2017). These receptors are a focus of both basic research and medical and biotechnology-centered studies.

Despite this attention there are holes in the potential GPCR target space. Most GPCRs are pharmacologically dark or currently undrugged, with only around 100 receptors uniquely targeted (Hauser et al. 2017). Additionally, some drugs cause adverse side effects that can be debilitating. Opioid receptor agonists are an example of this latter category, with many drugs causing decreased respiration and GI function in addition to pain relief (Raehal et al. 2005).

Most drug discovery technology relies on mammalian cell culture for initial screening assays (Hughes et al. 2011). There are major limitations with this approach. Cross talk between receptors is a known phenomenon that can make it challenging to know if the receptor of interest is targeted (Velloso et al. 1996; Prezeau et al. 2010). The number of potential downstream signaling pathways necessitates screens that rely on forced or native couplings of the receptors to these pathways, a strategy that is not always possible (Thomsen et al. 2005). Finally, mammalian cell culture is more expensive, slower and lower-throughput than other systems, limiting the feasibility of screening large numbers of compounds.

The yeast Saccharomyces cerevisiae has been used for roughly three decades to express human GPCRs and provides a work arounds for many of the limitations of mammalian-based screens. While humans and yeast had their last common ancestor over 1 billion years ago, they share several thousand genes, enabling use of the well-characterized and genetically-tractable yeast to study human proteins and systems (Kachroo et al. 2022; Kachroo et al. 2015). Yeast also have GPCRs, but just three, and two signaling pathways that are orthogonal to each other, the glucose sensing and pheromone response pathways (Versele et al. 2001) The yeast pheromone response pathway can be coopted to express heterologous GPCRs, providing a “null” background to study and manipulate these receptors.

Like human GPCRs, the native yeast pheromone GPCR, Ste2, responds to agonist, stabilizing an active conformation of the receptor that enables downstream signaling (Weis et al. 2018). This signaling is mediated by the separate G protein heterotrimer consisting of alpha, beta and gamma subunits. There are at least 18 G alphas in humans and receptors typically demonstrate preferences for coupling with certain subtypes (Hurowitz et al. 2000; Okashah et al. 2019). Yeast has a single G alpha, Gpa1, that signals with Ste2 (Versele et al. 2001). Upon agonist binding, guanosine diphosphate (GDP), bound to the G alpha subunit, is exchanged for guanosine triphosphate (GTP). This exchange catalyzes the dissociation of the beta and gamma subunits from the alpha subunit. Signaling cascades are then initiated from one or more of these un-trimerized subunits, of which there are many in humans, but just a single MAP kinase cascade coupled to Ste2 in yeast.

A number of modifications are necessary to enable the coupling of human GPCRs to the Ste2 G protein-mediated signaling pathway. The native receptor, Ste2, is knocked out, in addition to the proteins Sst2 and Far1, which increase sensitivity of the heterologous receptor's signaling and eliminate the cell cycle arrest response upon pheromone response pathway activation (Apanovitch et al. 1998; Alvaro et al. 2016). A reporter under control of the transcription factor Ste12, whose translocation is activated by the pheromone response pathway's MAP kinase cascade (Chen et al. 2007), can be utilized as a readout of heterologous GPCR activity in response to an agonist.

Additionally, chimeras of human G alpha proteins have been utilized to further improve signaling with heterologous receptors. The five amino acids at the C-terminus of Gpa1 are replaced with the corresponding human sub-type residues, enabling more efficient coupling of the G alpha and GPCR (Brown et al. 2000). While the unmodified Gpa1 has been shown to function in some cases (Mukherjee et al. 2015), usually human chimeras aid with effective signaling (Erlenbach et al. 2001). Typically, a native GPCR-G alpha chimera pair is used, but sometimes this pair does not produce the highest signal, highlighting the importance of screening a panel of multiple chimeras (Shaw et al. 2022; Lengger et al. 2022).

Over 50 human GPCRs have been expressed heterologously using these strategies. Yeast have been used as a tool to study the native receptors (Ladds et al. 2005) and as a chassis to study mutational effects, including the development of orthogonal GPCR-ligand pairs (Armbruster et al. 2007; Dong et al. 2010). The genetic tractability of yeast has enabled the discovery of new ligands (King et al. 1990; Price et al. 1995; Klein et al. 1998), including the deorphanization of receptors (Yasi et al. 2019).

Despite these achievements, expression and utilization of human GPCRs in yeast remains a challenge. Many receptors, particularly those of class B and C and odorant receptors (Lu et al. 2003), remain recalcitrant to functional expression and signaling in yeast. This recalcitrance can stem from several factors. Human receptors can become trapped in the endoplasmic reticulum (Erlenbach et al. 2001) and issues with trafficking can lead to a depletion of receptors at the cell membrane (O'Malley et al. 2009). The addition of leader sequences can sometimes improve trafficking problems and lead to increased signaling, but typically in receptor and leader-specific ways (Bean et al. 2022; Uddin et al. 2016; Iguchi et al. 2010). Differences in cell infrastructure between yeast and humans, like accessory proteins and membrane composition, can also affect expression and signaling (Bean et al. 2022; Weston et al. 2015).

Functional expression of human GPCRs in yeast remains a problem and general design strategies that enable expression and study of a wide range of GPCRs have yet to be determined. What is needed in the art are methods and compositions for cell expression libraries which can be used in a high throughput context.

### SUMMARY OF THE INVENTION

Disclosed herein is a non-naturally occurring test molecule for use in a cell-based screening method, wherein the molecule comprises: a molecule of interest comprising a message/address peptide; a linker sequence; and an anchor sequence which anchors the test molecule to a plasma membrane of the cell.

Also disclosed herein is a method of screening to determine interaction between a capture protein and a molecule of interest, the method comprising the steps of: providing the test molecule; providing a eukaryotic cell, wherein said eukaryotic cell comprises a non-endogenous capture protein; allowing the molecule of interest and the non-endogenous capture protein to interact; and determining interaction between the molecule of interest and the capture protein.

Further disclosed herein is a eukaryotic cell for use in a high throughput screening assay, wherein said cell comprises: one or more test molecules, wherein said test molecule is anchored to the cell's plasma membrane; and a capture protein, wherein said capture protein is found in the cell's plasma membrane, and is within proximity to the molecule of interest such that the molecule of interest and the capture protein can interact.

Disclosed herein is an expression vector encoding a protein of interest, wherein said protein of interest comprises a) a leader sequence which targets the protein for cell secretion; b) a molecule of interest coupled to a message/address peptide; c) a linker sequence; and d) an anchor sequence which anchors the molecule to a plasma membrane of the cell.

### BRIEF DESCRIPTION OF DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-B shows schematics of the native pheromone-response pathway and the modified pheromone response pathway for human GPCR expression. (A) To enable signaling with human GPCRs, the yeast GPCR Ste2 is knocked out in addition to Far1 and Sst2. A chimera of the yeast G alpha Gpa1 and the human subtype G alpha is used. (B) A GFP reporter is added under control of the transcription factor Ste12.

FIG. 2 shows dose responses with dynorphin A 1-8 and the human opioid receptors in different sterol backgrounds. KOR and DOR showed robust signaling in both the ergosterol and cholesterol backgrounds, while MOR showed minimal activity at only the highest concentrations. HsKOR, ERG EC₅₀=1.33 μM, 95% CI=0.768 to 2.43 μM; HsDOR, ERG EC₅₀=49.3 μM, 95% CI=32.7 to 86.4 μM; HsKOR, CHOL EC₅₀=3.71 μM, 95% CI=2.29 to 5.96 μM; HsDOR, CHOL EC₅₀=0.937 μM, 95% CI=0.674 to 1.30 M. The receptors were driven by pTDH3 and integrated into the yeast genome. For all experiments, n=3. Nonlinear regression was fit as a 4-parameter logistic equation.

FIG. 3 shows tethered peptide strategy. Peptides are expressed tethered to the truncated Flo1 protein, Flo42. The C-terminus of this protein encodes for a GPI anchor that localizes the peptide-protein complex to the plasma membrane, until the anchor is cleaved and Flo42 localizes to the inside of the cell wall. Localization of peptide agonists near their cognate GPCRs enables binding and signaling, triggering the MAP kinase cascade, and the expression of the GFP reporter.

FIG. 4 shows inducible expression of tethered dynorphin A 1-8 with the glycine-serine linker. Assays utilized the ergosterol-producing strain.

FIG. 5 shows expression cassette for the tethered peptide libraries. The MF alpha leader sequence targets the protein product for cell secretion. The library is randomized in either the first or last four residues of the eight amino acid dynorphin A 1-8 wild type peptide. The addition of the glycine-serine linker leads to greater GPCR signaling and downstream GFP expression. The Flo42 truncated protein localizes the peptides to the plasma membrane and cell wall. A galactose-inducible promoter is used to control expression.

FIG. 6 shows a heat map of amino acid enrichment for each position by the log proportion metric.

FIG. 7 shows library 1 variants by log proportion with the stringent nucleotide count filter.

FIG. 8 shows library 2 variants by log proportion with the stringent nucleotide count filter.

FIG. 9A-B shows enrichment of residues at each position with DOR by total nucleotide counts in the positive GFP pool. (A) shows DOR, library 1, and (B) shows DOR, library 2.

FIG. 10A-B shows enrichment of residues at each position with KOR by total nucleotide counts in the positive GFP pool. (A) shows KOR, library 1; (B) shows KOR, library 2.

FIG. 11A-B shows enrichment of residues at each position with MOR by total nucleotide counts in the positive GFP pool. (A) shows MOR, library 1; (B) shows MOR, library 2.

FIG. 12 shows Venn diagram of significant amino acid motifs for library 1 by the log proportion metric.

FIG. 13 shows Venn diagram of significant amino acid motifs for library 1 by the log proportion metric.

FIG. 14 shows logo map of variants in library 1 that are unique for individual receptors by the log proportion metric.

FIG. 15 shows logo map of variants in library 2 that are unique for individual receptors by the log proportion metric.

FIG. 16 shows logo map of variants in library 1 that are unique for individual receptors by the log proportion metric with the stringent read count filter.

FIG. 17 shows logo map of variants in library 2 that are unique for individual receptors by the log proportion metric with the stringent read count filter. DOR is not included because only the wild type motif, LRRI, met the read count threshold, and was not unique to this receptor.

FIG. 18 shows dose responses with exogenous dynorphin A 1-13 with KOR and DOR in both the ergosterol- and cholesterol-producing strains. Receptors were expressed on 2-micron plasmids.

FIG. 19 shows peptides had the long glycine-serine linker. There was no signaling with any of the N-termini anchored peptides, or the untethered peptides. Receptors were expressed on 2-micron plasmids, and tethered peptides constitutively on Cen6 plasmids.

FIG. 20 shows tethered peptide controls. Expression was driven by pGALI and integrated into the yeast genome. All data was in the cholesterol-producing strain, CJM263.

FIG. 21 shows Pearson correlation between technical replicates before filtering.

FIG. 22 shows Pearson correlation between technical replicates after filtering around a minimum log proportion of-11 and a maximum log proportion cutoff of-2.

FIG. 23 shows comparison of Pearson correlations before and after filtering.

FIG. 24 shows number of unique variant counts in each library and receptor pair for the GFP positive and GFP negative pools.

FIG. 25 shows variants with stop codons in the variable region. “False” means that the sequence has no stop codon, “true” means that it does. Within this grouping, number of variants in the negative and positive GFP populations are plotted.

FIG. 26 shows the tethering strategy for various linker and tether combinations.

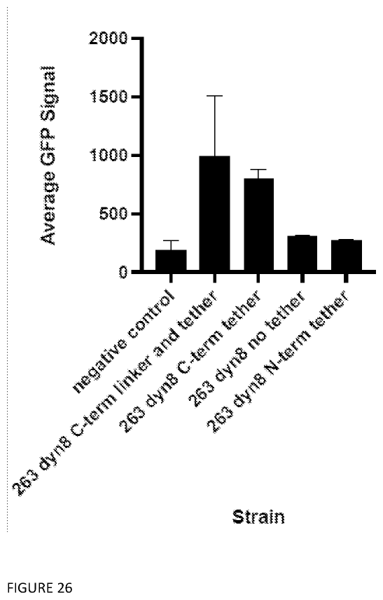

FIG. 27 shows two examples of isolates that signaled strongly and were unique to individual receptors. They were identified and cloned individually. The two examples shown are delta-specific peptides (YGMELRRI, SEQ ID NO: 12; and YGFDLLRI, SEQ ID NO: 13). For all of the receptors, the fifth residues appeared to be particularly important for activity and specificity.

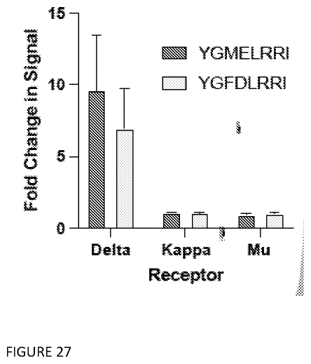

### DETAILED DESCRIPTION OF THE INVENTION

### Definitions

“Biological sample” as used herein is a sample of biological tissue or fluid. Such samples include, but are not limited to, tissue isolated from humans. Biological samples may also include sections of tissues such as frozen sections taken for histological purposes. A biological sample is typically obtained from a eukaryotic organism, such as insects, protozoa, birds, fish, reptiles, and preferably a mammal such as rat, mouse, cow, dog, guinea pig, or rabbit, and most preferably a primate such as chimpanzees or humans.

The phrase “functional effects” in the context of assays for testing compounds that modulate CB1R mediated activity includes the determination of any parameter that is indirectly or directly under the influence of the receptor, e.g., functional, physical and chemical effects. It includes ligand binding, changes in ion flux, membrane potential, current flow, transcription, G-protein binding, GPCR phosphorylation or dephosphorylation, signal transduction, receptor-ligand interactions, second messenger concentrations (e.g., cAMP, IP3, or intracellular Ca2+, in vitro, in vivo, and ex vivo and also includes other physiologic effects such increases or decreases of neurotransmitter or hormone release.

By “determining the functional effect” is meant assays for a compound that increases or decreases a parameter that is indirectly or directly under the influence of a receptor, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte receptor expression; tissue culture cell receptor expression; transcriptional activation of a receptor; ligand binding assays; voltage, membrane potential and conductance changes; ion flux assays; changes in intracellular second messengers such as cAMP and inositol triphosphate (IP3); changes in intracellular calcium levels; neurotransmitter release, and the like.

“Inhibitors,” “activators,” and “modulators” of a capture protein, such as a receptor, are used interchangeably to refer to inhibitory, activating, or modulating molecules identified using in vitro and in vivo assays for signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate activity, e.g., antagonists. Activators are compounds that, e.g., bind to, stimulate, increase, open, activate, facilitate, enhance activation, sensitize or up regulate activity, e.g., agonists. Modulators include compounds that, e.g., alter the interaction of a receptor with: extracellular proteins that bind activators or inhibitor; G-proteins; kinases; and arrestin-like proteins, which also deactivate and desensitize receptors. Modulators include genetically modified versions of a receptor, e.g., with altered activity, as well as naturally occurring and synthetic ligands, antagonists, agonists, small chemical molecules and the like. Such assays for inhibitors and activators include, e.g., expressing receptors in cells or cell membranes, applying putative modulator compounds, and then determining the functional effects of a molecule of interest on the receptor. Samples or assays comprising receptors that are treated with a potential activator, inhibitor, or modulator are compared to control samples without the inhibitor, activator, or modulator to examine the extent of inhibition. Control samples (untreated with inhibitors) are assigned a relative receptor activity value of 100%. Inhibition of receptor is achieved when the receptor activity value relative to the control is about 80%, optionally 50% or 25-0%. Activation of a receptor is achieved when the receptor activity value relative to the control is 110%, optionally 150%, optionally 200-500%, or 1000-3000% higher.

“Biologically active” capture protein refers to a capture protein, such as a receptor, having activity as described above, involved in interaction with a molecule of interest, such as a protein.

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

The term “reporter” is used herein in the broadest sense to describe a molecular entity, a characteristic and/or property of which (e.g., concentration, amount, expression, activity, cellular post-translational modification, localization, etc.) can be detected and correlated with a characteristic and/or property of a system containing the reporter (e.g., cell, artificial cellular entity, etc.). A “reporter” may be an intrinsic (e.g., endogenous) element of the system that exhibits one or more detectable and correlatable properties, or an artificial (e.g., exogenous) element engineered or introduced into the system (e.g., artificial cellular entity), that exhibits a detectable characteristic linked to process (e.g., gene expression) or component within the system. Suitable reporters include, but are not limited to: intrinsic genes or proteins (e.g., expression, concentration, activity, or protein-protein interactions of which may be correlated to a particular stimuli), exogenous genes or proteins (e.g., expression, concentration, activity, or protein-protein interactions of which may be correlated to a particular stimuli), luciferases, a beta lactamases, CAT, SEAP, a fluorescent proteins, etc.

As used herein the term “native receptor” refers to a ligand-binding protein of a cellular entity (e.g., located on the cell surface) that is also expressed by a non-engineered ancestral cell of the cellular entity. The native receptor on the cellular entity binds a ligand recognized or bound by the native receptor of the ancestral cell.

As used herein the term “non-native receptor” refers to a ligand-binding protein of an artificial cellular entity (e.g., located on the cell surface) that is not present in/on an ancestral cell of the artificial cellular entity. The non-native receptor on the artificial cellular entity typically binds a ligand not recognized or bound by native receptors of the ancestral cell. “Non-native receptors” may be receptors that are native to another cell type, a chimera of a native receptor and a receptor native to another cell type, a mutated native receptor (e.g., having various amino acid substitutions, deletions, and/or additions), an engineered receptor (e.g., a receptor that is not native to any cell), a chimera of a native receptor and an engineered receptor, etc.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, or chemical means. For example, useful labels include 32P, fluorescent dyes, electron-dense reagents, enzymes (e.g., as commonly used in an ELISA), biotin, digoxigenin, or haptens and proteins for which ant or 7 can be made detectable, e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al. Nuc. Acids Res. 25:3389-3402 (1977) and Altschul et al., J. Mol. Biol. 215:403-410 (1990), respectively. Software for performing BLAST analyses is publicly available through the National Center for Biotechnology Information (which can be found on the World Wide Web at ncbi.nlm.nih.gov). This algorithm involves first identifying high scoring sequence pairs (I-ISPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “selectively associates with” refers to the ability of a nucleic acid to “selectively hybridize” with another as defined above.

By “host cell” is meant a cell that contains an expression vector and supports the replication or expression of the expression vector. Host cells may be eukaryotic cells such as yeast, insect, amphibian, or mammalian cells such as CHO, HeLa and the like, e.g., cultured cells, explants, and cells in vivo.

### Methods and Compositions

The genetic tractability of yeast and specifics of its cellular physiology present advantages for drug discovery. Tethered agonism strategies, such as those disclosed in WO2019/228362A1 (herein incorporated by reference in its entirety for its teaching concerning tethered peptides), are known in the art. Disclosed herein are using those methods in screening assays, and molecules which can be used in said screening assays. Individual test molecules can be assayed in bulk using flow cytometry and can be sorted based on a fluorescent signal, for example. The genome of the sorted pools can then be extracted and the library-encoding region can be amplified and sequenced, such as with NGS, thereby linking phenotype to genotype.

Tethered agonism relies on the display of a protein in the periplasmic space (inside of the cell wall) of a eukaryotic cell, such as a yeast host cell. In some embodiments, the eukaryotic cell comprises one or more test molecules, and one or more capture proteins (such as a receptor) located in the periplasmic space of the yeast host cell. The test molecule and the capture protein are in proximity to each other, such that they can interact with each other. In some embodiments, the eukaryotic cell can be used to determine if the capture protein and the test molecule are able to interact.

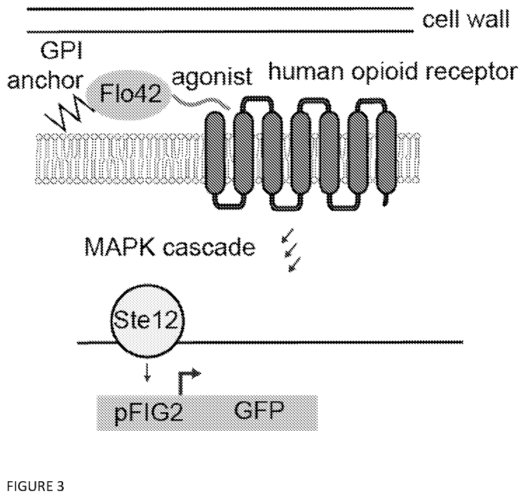

The test molecule to be displayed in the periplasmic space of the eukaryotic cell can be prepared by providing a molecule of interest comprising a message/address peptide; a linker sequence; and an anchor sequence which anchors the test molecule to a plasma membrane of the cell. Linkage can be covalent or noncovalent and is described in more detail below. Examples of the test molecule, and methods of using them, can be seen in FIGS. 3, 4, and 5.

### Test Molecules

Disclosed herein is a non-naturally occurring test molecule for use in a cell-based screening method, wherein the molecule comprises: a molecule of interest comprising a message/address peptide; a linker sequence; and an anchor sequence which anchors the test molecule to a plasma membrane of the cell.

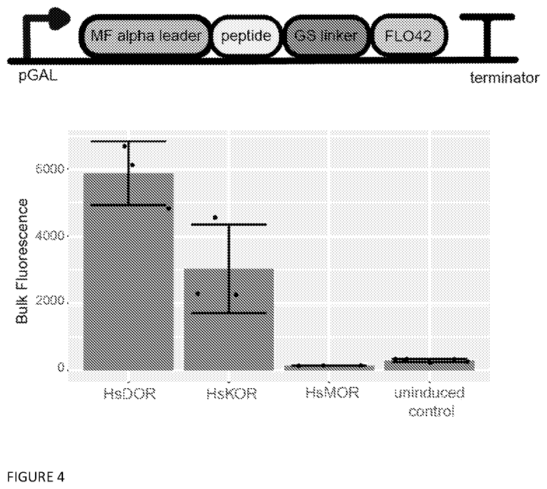

The test molecule comprises a molecule of interest, which is the actual portion of the test molecule which is to be tested for interaction with the capture protein. The molecule of interest can be selected from the group comprising a polypeptide, a peptide, a small molecule, a natural product, a peptidomimetic, a nucleic acid, a lipid, lipopeptide, or a carbohydrate, for example. The molecule of interest can be coupled to a message/address peptide, or in some circumstances, the message/address peptide can be the test molecule itself. An example of this can be seen in FIG. 4.

The message/address peptide is used to locate the test molecule to the desired area of the plasma membrane, such as near the desired capture protein. The “message” portion of the message/address peptide is used to make contact with the capture protein (such as a receptor) deep in a binding pocket of the receptor. This region is highly conserved between receptors, and is crucial for signaling. The “message” portion can be customized based on the desired capture protein. Due to the fact that messages are highly conserved, message portions of the peptide can be designed so that they can target the peptide to multiple receptors, for example.

The remaining portion of the message/address peptide residues are referred to as the “address.” This region contacts the receptor higher up the binding pocket, and is important for receptor-ligand specificity. In terms of the “address” region, there are not consensus residues at individual receptors that interact with the latter region of these agonists, indicating that variability is tolerated in this region. In a specific example, the message/address peptide can originate from, or be a derivative of, at least one of the enkephalin, dynorphin and endorphin peptides. When these peptides are used, the message portion of the message/address peptide can comprise the amino acids tyrosine-glycine-glycine-phenylalanine. Specific examples of such peptides can be found in SEQ ID NOS: 10-33 (Table 6).

The message/address peptide can be 4, 5, 6, 7, 8, 9, 10, 11, 12, 13, 14, 15, or 16 amino acids in length. In a preferred embodiment, the message/address peptide is 8 amino acids in length, wherein the first 4 amino acids are message, and the last four are the address. This is discussed in more detail in Example 1, and example motifs can be found in Table 1 and Table 6.

When the test molecule is a protein, the protein can further comprise a leader sequence which targets the test molecule for cell secretion. Examples of leader sequences include the MF alpha leader sequence, which is shown by way of example in SEQ ID NO: 7. Other leader sequences which can be used are known to those of skill in the art.

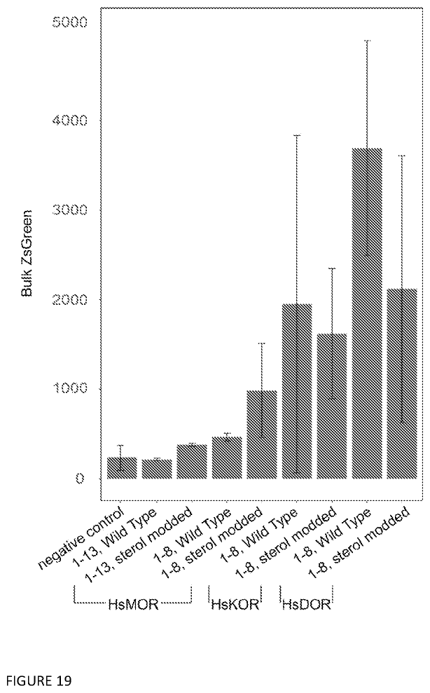

The linker sequence plays an important role in the test molecule. An example of a linker which can be used is a “glycine-serine linker,” wherein all, or a majority of, the residues comprising the linker are glycine and serine residues. In other words, the linker can consist of glycine and serine residues. The addition of a linker can enable increased movement of the peptides, allowing greater interaction with the receptor. FIGS. 4 and 19 show the importance of using a linker, and the effectiveness of glycine-serine-based linker. An example of a nucleic acid encoding a glycine-serine linker can be seen in SEQ ID NO: 9. The amino acid sequence of the glycine-serine linker can be GGGSGGGGSGGGSGGGGS (SEQ ID NO: 34). The linker can be 12, 13, 14, 15, 16, 17, 18, 19, 20, 21, 22, 23, or 24 amino acids in length, or longer, or shorter. In a preferred embodiment, the linker is 18 amino acids in length. Glycines and serines can make up 80, 85, 90, 95, or 100% of the linker. Amino acids other than glycine and serine can be used in the linker, such as alanine, arginine, asparagine, aspartic acid, cysteine, glutamic acid, glutamine, histidine, isoleucine, leucine, lysine, methionine, phenylalanine, proline, threonine, tryptophan, tyrosine, and valine.

The test molecule can also comprise an anchor sequence. In certain embodiments, the anchor sequence comprises a signal sequence that directs transport of the test molecule to the eukaryotic host cell periplasm, plasma membrane, or cell wall such that the test molecule is displayed in the periplasm. The anchor sequence can comprise a membrane-spanning transmembrane domain that projects the test molecule into the periplasm.

In certain embodiments, the anchor sequence comprises a cell-membrane associated protein domain that localizes to an external face of the cell membrane such that the displayed protein variant is projected into the periplasm. In certain embodiments, the periplasm anchor sequence is a protein that binds to an inner face of the cell wall such that the displayed test molecule is projected into the periplasm. In certain embodiments, the anchor sequence comprises a signal sequence that directs transport of the fusion protein to the eukaryotic host cell periplasm, and the anchor sequence is sufficiently large that the test molecule is retained in the periplasm. In certain embodiments, the anchor sequence is a component of a periplasmic protein complex that is sufficiently large that formation of the complex in the periplasm results in retention of the test molecule in the periplasm. In a specific example, the anchor sequence can comprise Flo1 or a derivative thereof, such as Flo42.

The components of the test molecule described above can be attached, or linked, to each other in various manners. For example, a molecule of interest may be linked covalently to an anchor sequence to form a fusion protein. Alternatively, a test molecule may form a complex with an anchor sequence, wherein the test molecule and the anchor sequence are linked noncovalently by molecular binding interactions in the complex. Alternatively, a test molecule and the anchor sequence can be linked covalently by a non-peptidic bond in a complex. In some embodiments, the non-peptidic bond is a disulfide bond. A protein to be displayed in the periplasmic space of the eukaryotic host cell can also be prepared by linking the protein to be displayed to a secretion signal.

### Methods of Detecting Interaction

Disclosed herein is a method of screening to determine interaction between a capture protein and a molecule of interest, wherein the molecule of interest is part of a test molecule. The test molecule for use in this method comprises a molecule of interest comprising a message/address peptide; a linker sequence; and an anchor sequence which anchors the test molecule to a plasma membrane of the cell. This “test molecule” is described in detail above, and examples of the test molecule can be found in Example 1.

The eukaryotic cell which is described herein can be yeast, insect, amphibian, or mammalian cells such as CHO, HeLa and the like, e.g., cultured cells, explants, and cells in vivo. In a preferred embodiment, the host cell is a yeast cell. The genus of the yeast host cell can comprise Saccharomyces, Candida, Pichia, Kluyveromyces, and Yarrowia. In some embodiments, the genus of the yeast host cells can be Saccharomyces. In some embodiments, the species of the yeast host cell is Saccharomyces cerevisiae. In one embodiment, 1, 2, 3, 4, 5, 6, 7, 8, 9, or 10 or more genes of the host cell that encode undesirable cellular functions or responses of the host cell can be disabled or replaced. For example, the cell cycle arrest genes can be knocked out.

The eukaryotic host cell comprises a non-endogenous capture protein. Examples of the non-endogenous capture protein include, but are not limited to, a receptor, an ion channel, and a transporter. In a specific example, the receptor can be a G-coupled protein receptor (GPCR). More specifically, the GPCR can be a cannabinoid receptor, such as CB1R or CB2R. In some embodiments, the capture protein can comprise one or more mutations that affect its binding activity.

In this method, the capture protein and the molecule of interest are allowed to interact, meaning that they are placed under sufficient conditions such that they are allowed to come into contact with each other. For example, both the molecule of interest and the capture protein can both be located in the plasma membrane. The message/address portion of the test molecule would have placed it within proximity of the capture protein, such that they are within a reasonable distance from each other in order to interact. One can then determine this interaction between the molecule of interest and the capture protein. This interaction can be determined by various means which are discussed below.

Polynucleotides encoding the test molecule, and/or the polynucleotides encoding the capture protein, can be provided by expression vectors. In other embodiments, they can be integrated into the yeast host cell genome at a target locus. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid encoding a polynucleotide of interest is under transcriptional control of a promoter. A “promoter” refers to a DNA sequence recognized by the synthetic machinery of the cell, or introduced synthetic machinery, required to initiate the specific transcription of a gene. The term promoter is used herein to refer to a group of transcriptional control modules that are clustered around the initiation site for RNA polymerase I, II, or III. Enhancer elements may be used in association with the promoter to increase expression levels of the constructs. In certain embodiments, an expression vector comprises a promoter “operably linked” to a polynucleotide encoding a test molecule or a capture protein. The phrase “operably linked” or “under transcriptional control” as used herein means that the promoter is in the correct location and orientation in relation to a polynucleotide to control the initiation of transcription by RNA polymerase and expression of the test molecule or the capture protein.

Typically, transcription terminator/polyadenylation signals will also be present in the expression construct. Examples of such sequences include, but are not limited to, those derived from SV40, as described in Sambrook et al, supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22 150-161. A multitude of IRES sequences are known and include sequences derived from a wide variety of viruses, such as from leader sequences of picomaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100 (25): 15125-15130), an IRES element from the foot and mouth disease virus (Ramesh et al., Nucl. Acid Res. (1996) 24:2697-2700), a giardiavirus IRES (Garlapati et al, J Biol. Chem. (2004) 279 (5): 3389-3397), and the like. A variety of nonviral IRES sequences will also find use herein, including, but not limited to IRES sequences from yeast, as well as the human angiotensin II type 1 receptor IRES (Martin et al, Mol. Cell Endocrinol. (2003) 212:51-61), fibroblast growth factor IRESs (FGF-1 IRES and FGF-2 IRES, Martineau et al. (2004) Mol. Cell. Biol. 24 (17): 7622-7635), vascular endothelial growth factor IRES (Baranick et al. (2008) Proc. Natl. Acad. Sci. U.S.A. 105 (12): 4733-4738, Stein et al. (1998) Mo/. Cell. Biol. 18 (6): 3112-3119, Bert et al. (2006) RNA 12 (6): 1074-1083), and insulin-like growth factor 2 IRES (Pedersen et al. (2002) Biochem. J. 363 (Pt 1): 37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a test molecule for display in combination with capture protein from an expression cassette.

Alternatively, a polynucleotide encoding a viral T2A peptide can be used to allow production of multiple protein products from a single vector. 2A linker peptides are inserted between the coding sequences in the multicistronic construct. The 2A peptide, which is self cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLOS One 6 (4): e 18556, Trichas et al. (2008) BMC Biol. 6:40, Provost et al. (2007) Genesis 45 (10): 625-629, Furler et al. (2001) Gene Ther. 8 (11): 864-873; herein incorporated by reference in their entireties.

In certain embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leul+, ade6+), antibiotic selection markers (e.g., aphAl or ble), fluorescent markers (e.g., mCherry, green fluorescent protein), bioluminescent markers (e.g., luciferase), or other markers for selection of transformed yeast cells. The yeast plasmid may further contain components to allow shuttling between a bacterial host (e.g., E. coli) and yeast cells. A number of different types of yeast plasmids are available including yeast integrating plasmids (Yip), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

Inclusion of regulatory sequences may also be desirable, which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. For example, a pheromone-inducible promoter, such as a PRM1 or FUS2 promoter can be used to make transcription dependent on activation of the pheromone signaling pathway. The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et ah, supra; DNA Cloning, Vols. I and II, supra; Nucleic Acid Hybridization, supra.

In one embodiment, recombinant polynucleotides encoding protein variants are cloned into a periplasm-targeting expression vector comprising: a) a polynucleotide encoding a signal peptide; b) a cloning site suitable for in-frame insertion of a polynucleotide encoding a test molecule after the polynucleotide encoding the signal peptide; c) a polynucleotide encoding a glycophosphatidylinositol (GPI) plasma membrane anchoring domain, positioned such that the vector is capable of producing a fusion protein comprising the signal peptide and the test molecule fused to the GPI plasma membrane anchoring domain; and d) a promoter operably linked to sequences encoding the fusion protein.

In some embodiments, an affinity tag, epitope, label, or the like, is added to the test molecule to allow measurement of the total display level in host cells. As used herein, the term “affinity tag” refers to a biomolecule, such as a polypeptide segment, that can be attached to a second biomolecule to provide for purification or detection of the second biomolecule or provide sites for attachment of the second biomolecule to a substrate. Examples of affinity tags include a poly-histidine tract, protein A (Nilsson et al. (1985) EMBO J. 4:1075; Nilsson et al. (1991) Methods Enzymol. 198:3, glutathione S transferase (Smith and Johnson (1988) Gene 67:31), Glu-Glu affinity tag (Grussenmeyer et al., (1985) PNAS USA 82:7952), substance P, FLAG peptide (Hopp et al. (1988) Biotechnology 6:1204), streptavidin binding peptide, or other antigenic epitope or binding domain, and the like, (Ford et al. (1991) Protein Expression and Purification 2:950), all of which are herein incorporated by reference. As used herein, a “label” is a molecule or atom which can be conjugated to a biomolecule to render the biomolecule or a form of the biomolecule, such as a conjugate, detectable or measurable. Examples of labels include fluorescent agents, bioluminescent proteins, photoactive agents, radioisotopes, paramagnetic ions, chelators, and the like.

In order to effect expression of sense or antisense gene constructs, the expression construct must be delivered into a host cell. This delivery may be accomplished in vitro using laboratory procedures for transforming yeast cells well-known in the art, such as spheroplast transformation, alkaline ion treatment (e.g., Cs⁺ or Li⁺), electroporation, trans-kingdom conjugation, electroporation, and biolistic and glass bead methods (see, e.g., Kawai et al. (2010) Bioeng. Bugs. 1 (6): 395-403, Gietz et al. (1995) Yeast 11 (4): 355-360, Gietz et al. (2007) Nat. Protoc. 2 (1): 38-41, Hinnen et al. (1978) Proc. Natl. Acad. Sci. USA 75:1929-1933, Avery et al. (1995) Mol. Med. 1 (4): 344-365, Ito et al. (1983) J. Bacteriol. 153:163-168, Johnston et al. (1988) Science 240:1538-1541, Dohmen et al. (1991) Yeast 7 (7): 691-246, Hayama et al. (2002) J. Biosci. Bioeng. 94 (2): 166-171, and Wang et al. (2001) Crit. Rev. Biotechnol. 21 (3): 177-218; herein incorporated by reference).

Once an expression construct has been delivered into the cell, the nucleic acid encoding the gene of interest may be positioned and expressed at different sites. In certain embodiments, the nucleic acid encoding the gene may be stably integrated into the genome of the cell via homologous recombination. This integration may be in the cognate location and orientation (gene replacement), within a gene (gene disruption), or in a random, non-specific location (gene augmentation). Integration of a construct at a target locus that disrupts a gene may be acceptable as long as the gene disruption does not interfere with cell growth or screening of the yeast periplasmic display library (e.g., avoid disruption of pheromone response if used in screening).

In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments, the expression construct may simply consist of naked recombinant DNA or plasmids. Transfer of the construct may be performed by any of the methods mentioned above which physically or chemically permeabilize the cell membrane.

In still another embodiment, a naked DNA expression construct may be transferred into cells by particle bombardment. This method depends on the ability to accelerate DNA-coated microprojectiles to a high velocity allowing them to pierce yeast cell walls and membranes and enter cells without killing them (Armaleo et al. (1990) Curr. Genet. 17 (2): 97-103). Several devices for accelerating small particles have been developed. One such device relies on a high voltage discharge to generate an electrical current, which in turn provides the motive force (Y ang et al. (1990) Proc. Natl. Acad. Sci. USA 87:9568-9572). The microprojectiles may consist of biologically inert substances, such as tungsten or gold beads.

In some embodiments, a collection of linear DNA molecules encoding test molecules are generated. Rather than cloning the linear DNA molecules into a vector prior to transformation, the host cells are transformed with an empty vector together with the collection of linear DNA molecules encoding the test molecules, which subsequently integrate into the vector in vivo, e.g., by homologous recombination in the yeast host cells.

When the capture protein is a GPCR, such as a cannabinoid receptor (for example, CB1R or CB2R), the native Ste2 and/or Ste3 of the host yeast cell can be replaced with a heterologous GPCR, wherein said heterologous GPCR has a truncated N terminus of at least 5 residues; and further wherein native G alpha protein (Gpa1) has been replaced with a gene encoding a chimeric Gpa1.

### High Throughput Screening Methods

The methods disclosed herein can be used as a high-throughput screen to identify test compounds which interact with the capture protein. In this case, the test molecule can be labeled. The terms “label” and “detectable label” refer to a molecule capable of detection, including, but not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to radiolabels (e.g., H, I, S, C, or P), stable (non radioactive) heavy isotopes (e.g., 13C or 15N), phycoerythrin, fluorescein, 7-nitrobenzo-2-oxa-1,3-diazole (NBD), YPet, CyPet, Cascade blue, allophycocyanin, Alexa dyes (e.g., Alexa 350, Alexa 430, Alexa 488, Alexa 532, Alexa 546, Alexa 555, Alexa 594, Alexa 647, Alexa 660, Alexa 680, and Alexa 750), Atto dyes (e.g., Atto 488, Atto 532, Atto 550, Atto 565, Atto 590, Atto 610, Atto 620, Atto 635, Atto 647, Atto 655, and Atto 680), cyanine dyes (e.g., Cy3, Cy5, and Cy7), TYE 563, TYE 665, TYE 705, TEX 615, JOE, TET, HEX, TAMRA, ROX, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenicol acetyl transferase, and urease. Enzyme tags are used with their cognate substrate. As with many of the standard procedures associated with the practice of the invention, skilled artisans will be aware of additional labels that can be used.

For example, interaction of the test molecule with the capture protein can trigger expression of the reporter. This can be done by way of a signaling cascade, such as the MAP kinase cascade, which is pictured in FIG. 3. In this case, test molecules that elicit a signaling pathway can be selected for expression of a reporter gene placed under the control of a specific promoter that is activated by the signaling pathway. Reporter genes used in this type of selection can be, e.g., genes conferring a detectable physical attribute (e.g., GFP and beta-lactamase) or antibiotic resistance (e.g., aminoglycoside phosphotransferase) to the cell. Specifically, the signal can be a fluorescent one.

More specifically, some of the reporting systems that can be used with the biosensors disclosed herein include, but not limited to, fluorescence (GFP, RFP, YFP, ZsGreen), luminescence (Lux, Luc), colorimetric (beta-galactosidase), electrical, and growth (His3, Trp1, and Leu2). Also contemplated herein is the use of metal nanoparticles, such as gold and silver, in biosensing. The eukaryotic host cell can be engineered to express a reporter of expression of the capture protein.

Exemplary methods for evaluating phenotypes of cells include microscopy (e.g., light, —confocal, fluorescence, scanning electron, and transmission electron), fluorescence based cell sorting, differential centrifugation, differential binding, immunoassays, enzymatic assays, growth assays, and in vivo assays. In some embodiments, phenotypic behaviors of the cell such as chemotaxis, morphological changes, or apoptosis can be monitored via visual inspection or microscope examination. Optionally, computer software programs can be used to automatically detect cells with altered phenotype. To this end, various high-content screens (“HCS”) have been developed to address the need for more detailed information about the temporal-spatial dynamics of cell constituents and processes. High-content screens automate the extraction of multicolor fluorescence information derived from specific fluorescence-based reagents incorporated into cells (see, e.g., Giuliano and Taylor, Curr. Op. Cell Biol. 7:4, 1995). Cells can be analyzed using an optical system that can measure spatial, as well as temporal dynamics. In addition, many fluorescent physiological indicators and “biosensors” are available to monitor changes in biochemical and molecular activities within cells (see, e.g., Giuliano et al., Ann. Rev. Biophys. Biomol. Struct. 24:405, 1995). By way of specific example, flow cytometry can be used.

The test molecules disclosed herein can comprise a library of test molecules, such that 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1×10⁶, 1×10⁷, 1×10⁸, 1×10⁹, 1×10¹⁰ or more test molecules are present in a given assay. These can be present in more than one cell, such that 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1×10⁶, 1×10⁷, 1×10⁸, 1×10⁹, 1×10¹⁰ or more cells are present in an assay.

The test molecules can be assayed to determine their function, meaning whether or not they are capable of binding the capture protein. This can be done in one round, or over multiple rounds of selection, such as 2, 3, 4, 5, 6, 7, 8, 9, 10, 15, 20, 30, 40, 50, or more rounds. The multiple rounds of selection can be done using FACS or culture growth. In each round, a cell population can be re-started following a previous round of FACS, growth, or other means of selection known to those of skill in the art.

### Biosensors and Platforms

Disclosed herein is a biosensor comprising the engineered eukaryotic cell described herein. The unique difficulties of engineering biosensors in yeast is described in Adeniran et al. (Adebola Adeniran, Michael Sherer, Keith E. J. Tyo, Yeast-based biosensors: design and applications, FEMS Yeast Research, Volume 15, Issue 1, February 2015, Pages 1-15, hereby incorporated by reference in its entirety). The modifications described above are key to the success of the specific engineered biosensors described herein. A wide variety of reporting systems can be used with the biosensors disclosed herein, including, but not limited to, fluorescence (GFP, RFP, YFP, ZsGreen), luminescence (Lux, Luc), colorimetric (beta-galactosidase), electrical, and growth (His3, Trp1, and Leu2). Also contemplated herein is the use of metal nanoparticles, such as gold and silver, in biosensing.

In some embodiments, the capture protein-expressing cells disclosed herein are used as biosensors within a system or device (e.g., POC system/device) configured for the detection of one or more analytes (test molecules) in a sample. Contemplated herein are one or more of: reagents (e.g., buffers, etc.) storage, sample purification, introduction of the sample and biosensors, mixing, reaction, signal detection, signal quantification, communication of results (e.g., on a screen, on a printer report, etc.), etc. A system/device may be of any suitable configuration for carrying out the particular detection/quantification assay. A system/device may comprise a single unit, or multiple modules (e.g., regent module, mixing module, reaction module, detection module, etc.). In particular embodiments, a system/device is configured for point-of-care applications or research applications. Exemplary systems/devices, all or portions of which may find use in embodiments herein, are described, for example, in: U.S. Pat. No. 8,697,377; WO 2014/134537; U.S. Pat. No. 7,604,592; U.S. Pat. Pub. 2013/0210652; U.S. Pat. Pub. 2014/0320807; U.S. Pat. Nos. 8,523,797; 8,005,686; 8,283,155; 8,110,392; each of which is herein incorporated by reference in their entireties.

The biosensors described herein may find use is any suitable field. In medicine, devices/systems incorporating the capture proteins described herein find use, for example: in hospitals and medical clinics for bedside/in-room detection of biomarkers (e.g., for quick and reliable detection/diagnosis of disease, pathogen, condition, etc.); for in-the-field detection of pathogens or diagnosis; etc. In research, the biosensors herein find use, for example, in high throughput screening to search libraries of mutant proteins. These uses are discussed in more detail below. The applications/uses described herein are not limiting.

Also disclosed herein is a platform for the creation of yeast-based biosensors for capture proteins. The structure of the receptor is highly evolvable, making it amenable to detecting multiple ligands, both naturally and non-naturally occurring.

Further disclosed is a biosensor for the detection of compounds which interact with one or more capture proteins. Either one or more capture proteins may be expressed in a cell. Either one or both of these receptors may be genetically engineered, as described elsewhere herein.

In the biosensors and methods of using them described herein, the analyte, or test molecule, can be labeled. The biosensor can be high-throughput. Examples of high throughput systems using yeast biosensors can be found in Qiu et al. (Qiu C, Zhai H, Hou J. Biosensors design in yeast and applications in metabolic engineering. FEMS Yeast Res. 2019 Dec. 1; 19 (8): foz082, herein incorporated in its entirety for its teaching regarding yeast biosensors). When the system is designed as high-throughput, a library of analytes can be used to screen for analyte-receptor interaction.

When more than one capture protein is present in the same cell, a differential binding assay can be used to determine if a test molecule preferentially binds one capture protein over the other. By “preferentially bind” is meant that a certain compound binds to one receptor or the other with one receptor over the other by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 2, 3, 4, 5, 6, 7, 8, 9, or 10 fold or more, or any amount above, below, or in-between these values.

The methods disclosed herein can be used to quantify the expression of a target molecule. For example, one can carry out a quantification assay via cell sorting and flow cytometry. In one method, the cells can first be sorted into two pools, “off” and “on” via gating around a negative control, so any cell with a true green fluorescent signal would be called as positive. The more true quantification happens via testing of individual isolates identified via this cell sorting and NGS. Individual test molecules can be cloned and tested on a flow cytometer. Here, the fluorescent signal can be measured for 10,000 or more cells, and fold change can be calculated via signal over the background of the negative control. With this strategy one can quantify test molecules in relations to other test molecules. For example, one may have 2, 3, 4, 5, 6, 7, 8, 9, 10, 11, 12, 13, 14, 15, 16, 17, 18, 19, 20, 21, 22, 23, 24, 25, 26, 27, 28, 29. 30, or more fold change in signal, while the other test molecule has a significantly different fold change. By way of specific example, one may have a fold change of 20, while the other only has a fold change of 2. In this way, the amount or sequence of an expressed mRNA of the test molecule can be determined.

### Cells, Proteins, and Nucleic Acids

Disclosed herein are peptides comprising any of SEQ ID NOS: 10-33. Also disclosed are peptides wherein 1, 2, 3, or 4 amino acids of any one of SEQ ID NOS: 10-33 have been replaced. Also disclosed herein are SEQ ID NOS: 1-9, which are nucleic acids encoding various components of an engineered opioid receptor, as well as various components of a test molecule. Also disclosed are those proteins encoded by SEQ ID NOS: 1-9. Also disclosed are nucleic acids with 80%, 85%, 90%, 95%, 96%, 97%, 98%, or 99% identity to SEQ ID NOS: 1-9, or to the proteins which are encoded by these nucleic acids.

Also disclosed herein are eukaryotic host cells for use in a high throughput screening assay, wherein said cell comprises: one or more molecules of interest, wherein said molecule of interest comprises the non-naturally occurring test molecule described above, wherein said test molecule is anchored to the cell's plasma membrane; and a capture protein, wherein said capture protein is found in the cell's plasma membrane, and is within proximity to the molecule of interest such that the molecule of interest and the capture protein can interact. The anchor sequence can comprise a membrane-spanning transmembrane domain or a membrane associated protein domain that projects the test molecule into the cell's periplasm. The anchor sequence can be a protein that binds to an inner face of the cell wall such that the molecule of interest is projected into the cell's periplasm.

Further disclosed is an expression vector encoding a protein of interest, wherein said protein of interest comprises a) a leader sequence which targets the protein for cell secretion; b) a molecule of interest coupled to a message/address peptide; c) a linker sequence; and d) an anchor sequence which anchors the molecule to a plasma membrane of the cell. Also disclosed is a nucleic acid encoding this expression vector, and a cell comprising the expression vector.

### EXAMPLES

### Example 1: Identification of Opioid Receptor Agonists Using Self-Produced Peptide Libraries in Yeast

G protein-coupled receptors (GPCRs) are a large class of membrane proteins that encompass a large percentage of drug targets. Signaling networks from GPCR activation are complex and often drug and context specific, making development of drug discovery screens challenging. The “null” background of the yeast Saccharomyces cerevisiae provides a simplified platform to screen human receptors. In addition to simplified signaling, the genetic tractability and cellular physiology of yeast enable tethering of peptides to the inside of the yeast cell wall in an orientation able to activate heterologous GPCRs. This strategy allows the compartmentalized screening of hundreds of thousands of peptides for their ability to agonize receptors. Demonstrated herein is the use of this strategy for the first time as a screening tool for GPCRs. In starting with a non-specific endogenous opioid peptide a library of peptide mutants were created and screened for those that activated each of the opioid receptor subtypes, finding general amino acid trends that inform an understanding of ligand-receptor dynamics, and peptide leads that are unique for receptor sub-type.

### Introduction

G protein-coupled receptors (GPCRs) are membrane proteins that respond to a variety of external stimuli and trigger signal transduction inside of the cell when bound to ligand. There are over 800 human GPCRs and they are extremely important pharmacologically-roughly 34% of all FDA-approved drugs target a GPCR (Hauser et al. 2017). Despite this percentage, there are holes in the potential GPCR target space. Most GPCRs are pharmacologically dark or currently undrugged, with only around 100 non-olfactory receptors targeted (Hauser et al. 2014). Additionally, there is a need for drugs without adverse side effects, which can often be debilitating. Opioid drugs are an example of this phenomenon, with many drugs causing decreased respiration and GI function in addition to pain relief (Benyamin et al. 2008).

Most drug discovery technology relies on mammalian cell culture to screen for candidates. There are major limitations with this approach. Cross talk between receptors can make it challenging to determine if the receptor of interest independently causes the assay's output. Additionally, the number of potential downstream signaling pathways necessitates screens that rely on forced or native couplings of the receptors to these pathways, strategies that are not always possible. Finally, mammalian cell culture is more expensive, slower and lower-throughput than other systems, limiting the feasibility of very large compound screens.

The yeast Saccharomyces cerevisiae provides an alternative drug discovery platform. Yeast have just three GPCRs and two GPCR signaling cascades—the pheromone response pathway and the glucose sensing pathway, which are orthogonal to each other. For decades, researchers have modified the pheromone response pathway to enable signaling with human GPCRs. In addition to other genetic modifications, a reporter, often a GFP, is driven by a transcription factor activated by the pheromone response pathway¹⁸. When an agonist binds to the GPCR of interest, GFP is expressed, which can then be read using flow cytometry. The “null” background of this system provides a relatively simple, fast and inexpensive way to screen compounds against GPCRs.

Beyond simplified signaling cascades, the genetic tractability of yeast and specifics of its cellular physiology present additional advantages for drug discovery. Researchers have built on the long history of heterologous peptide and protein production and secretion in yeast (Porro et al. 2005) to develop a tethered agonism strategy to display peptides on the inside of the cell wall in a compartmentalized autocrine fashion (Ishii et al. 2012). Peptides are expressed from a genetic cassette, located on a plasmid or integrated in the yeast genome. A signal sequence is used to localize the peptide towards the plasma membrane (Martoglio et al. 1998), and a truncated cell wall protein, Flo1, tethers the agonist to the inside of the cell wall. This orientation allows the peptide to interact with GPCRs in the plasma membrane, but not with surrounding cells, ensuring that the agonist is tied to the gene that encodes it.

Tethered peptide agonism has been used with individual peptide agonists, but not to screen libraries of peptides against GPCRs. A common strategy for the development of peptide therapeutics that target GPCRs is modification of the endogenous peptide sequence (Davenport et al. 2020). Variant screening of endogenous peptide agonists in the tethered format could enable high-throughput analysis of many variants at a low-cost. Due to the ease of genetic manipulation, libraries of 108 unique peptides can be screened in yeast using flow cytometry (Goodnow et al. 2017). The cost of this type of screen would be several hundred dollars, compared to the $0.4-2 billion to synthesize and screen a 1 million compound library exogenously (Goodnow et al. 2017), in addition to the time and resources to manually compartmentalize these candidates. Peptide drugs, while a small share of the drug market space, are increasingly being investigated as drug candidates, including as GPCR agonists (Muttenhaler et al. 2021). While peptide therapeutics have traditionally been limited by poor pharmacokinetics and oral bioavailability, recent advances have made progress to address these challenges.

Receptors of particular importance for drug discovery are the opioid receptors, μ (MOR), κ (KOR), and δ (DOR). The opium poppy, Papaver somniferum, has been used by humans for over 5,000 years for its active ingredients, including morphine, that provide pain relief (Pthan et al. 2012). Over the last few hundred years, natural, semi-synthetic, and synthetic opioids have been utilized with increasing efficacy and precision to treat pain. All three receptors regulate pain and pleasure broadly, with MOR regulating acute pain, and DOR chronic pain (Quirion et al. 2020). Most current opioid drugs act primarily at MOR, which, while effective at providing analgesia, typically have negative side effects, including decreased respiration which can result in death. New MOR drugs that activate certain downstream signaling cascades over others could be useful for evading these negative effects (Viscusi et al. 2016). Activation of the KOR often causes dysphoria (Valentino et al. 2018); however, it has been hypothesized that KOR-selective agonists could provide the pain relief of MOR agonists without the negative side effects (O'Connor et al. 2015; Che et al. 2018). In addition to pain, KOR agonists have the potential to regulate mood disorders and addiction (Bruchas et al. 2010; Bruchas et al. 2016). Development of opioid agonists that provide pain relief, and potentially treatment of other conditions, without negative side effects could be a significant way to address the current epidemic of death and overdose from opioid abuse (Manchikanti et. Al 2012).

In addition to exogenous opioids used to treat pain, the opioid receptors are regulated by endogenous opioid peptides. These peptides are all cleaved from three precursors—proenkephalin, prodynorphin, and proopiomelanocortin (Fricker et al. 2020). While nonspecific for individual receptors, these ligands typically demonstrate preference for receptor subtype (Fricker et al. 2020). Crucially, the enkephalin, dynorphin and endorphin peptides all share the same first four amino acids of tyrosine-glycine-glycine-phenylalanine. These residues, commonly referred to as the “message” of the peptide, contact the receptor deep in the binding pocket. This region is highly conserved between the receptors, and is crucial for signaling (Granier et al. 2012). The latter peptide residues are referred to as the “address.” This region contacts the receptor higher up the binding pocket, and is important for receptor-ligand specificity (Portoghese et al. 1989; Cheng et al. 2018).

While the message-address concept of endogenous peptide activation has been paradigm for decades, evidence has accumulated that there is more flexibility in these definitions than previously thought. The phenolic hydroxyl of the tyrosine in position 1 appears to be crucial for receptor activation (Lai et al. 2006), and similar moieties are present in non-peptide opioid agonists and antagonists. But beyond this first position, it remained unclear how important residues 2-4 are for signaling, and how much residue variability is tolerated in these positions. Additionally, at least some modifications in this “message” region affect specificity, not just generalized activity (Lung et al. 1996), and this region has also shown to be more flexible than previously thought, moving significantly in the KOR binding pocket. In terms of the “address” region, there are not consensus residues at individual receptors that interact with the latter region of these agonists, indicating that variability is tolerated in this region (Vardy et al. 2013). A deep analysis of how each residue of any of the endogenous opioid peptides affects opioid receptor function has not been executed until now.

In this study an endogenous opioid peptide scaffold was used to create libraries of tethered mutagenized peptides in yeast in either the “message” or “address” regions. By sorting cells based on the GFP output of the pheromone response pathway, potential new ligands were identified for the three opioid receptors. General residue and motif trends were also found for these receptors, increasing knowledge of ligand binding dynamics at these receptors.

### Results

### Development of Opioid Receptor Biosensors

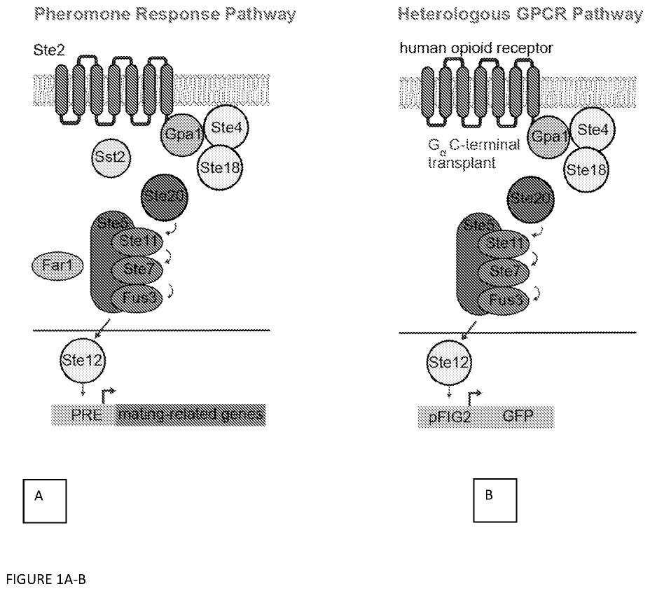

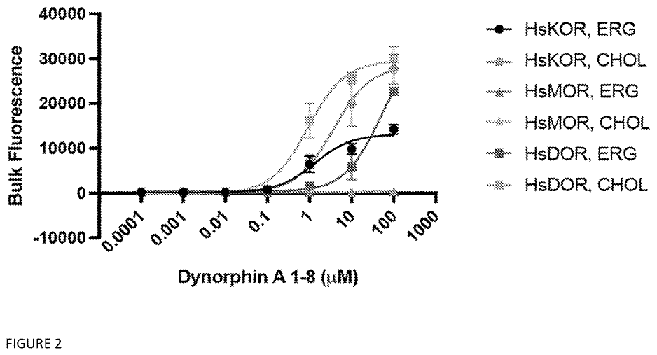

To express the human opioid receptors in yeast, modified strains previously developed were used (Bean et al. 2022). These strains have Ste2, the native yeast mating GPCR, knocked out, in addition to Sst2 and Far1. Sst2 deletion increases the signal to noise response by decreasing receptor desensitization, and the Far1 deletion disables the cell cycle arrest response upon GPCR activation. ZsGreen was added behind the FIG. 2—Ste12 inducible promoter for GFP-expression upon heterologous GPCR-activation. The strains were additionally modified with the replacement of the five residues of the C-terminal native yeast G alpha, Gpa1, with the human Gᵢₐ₃ residues. This G alpha sub-type is known to couple with the human opioid receptors and has been demonstrated previously in yeast (Bean et al. 2022) (FIG. 1).

Two of the three endogenous opioid peptide types were previously tested against the three opioid receptors in yeast (Uddin et al. 2016). Both met-enkephalin and dynorphin A 1-17 showed activity against MOR, DOR, and KOR. None of the endorphins were tested. To determine which peptide to use as a scaffold for the tethered libraries, the number of residues was considered. The ideal scaffold would provide an “address” region long enough to find agonists unique to receptor subtype, but short enough to cover the mutational space of the residues. The enkephalin peptides are five amino acids, with either a leucine or methionine after the conserved YGGF²³⁵. This length is potentially too short for effective tethered activation, and variants would consist primarily of the “message” region. The length of the endorphins, 16 residues at the shortest and 31 at the longest (Kimura et al. 1980), provides more mutational space than the ability to fully cover in yeast, necessitating a library of 2.56×10¹⁰ variants. The dynorphin A peptides are of a more moderate length between eight and seventeen amino acids. The shortest, dynorphin A 1-8, is less sub-type specific than the longer versions (Schwarzer et al. 2009), potentially providing a good scaffold. The eight amino acids are a more manageable mutational space with the potential for saturation in a yeast-based library.

The human opioid receptors have been expressed previously in a cholesterol-producing background. Humans produce cholesterol as the dominant sterol in their plasma membranes, and it is known that sterol-GPCR interactions are important for receptor signaling and conformational stability (Sarkar et al. 2022). Ergosterol is the dominant sterol in the yeast plasma membrane (Parks et al. 1995). It was previously demonstrated that signaling with MOR was more sensitive and had a higher dynamic range in the strain with cholesterol. While the opioid receptors signaled robustly in the cholesterol-background, the strain had a growth defect compared to wild-type. To address the potential that this growth defect could preclude library transformation efficiency, the receptors were assayed in both wild type ergosterol- and cholesterol-producing backgrounds with the peptides dynorphin A 1-8 and dynorphin A 1-13.

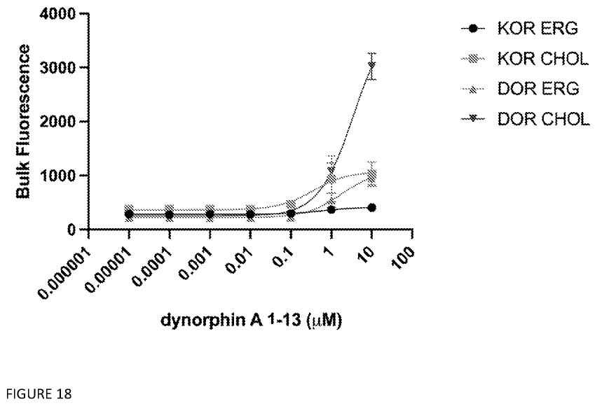

DOR and KOR showed robust signaling with dynorphin A 1-8 in both backgrounds, while MOR showed activity at only the highest concentrations in both strains (FIG. 2). This result was surprising, as the longer and more KOR-biased peptide, dynorphin A 1-17, had a higher fold change and a pEC₅₀ of 5.8 in the cholesterol-producing strain. One difference between expression strategies in these studies is that the receptors were previously expressed on 2-micron plasmids and integrated into the yeast genome for this work. Expression of GPCRs on 2-micron plasmids almost certainly leads to more receptor at the plasma membrane, but copy number can vary dramatically between cells, making fluorescence comparisons between cells during flow cytometry challenging (Lee et al. 2015). The longer dynorphin A 1-13 showed much less activity than the shorter peptide (FIG. 18). Due to the mixed signaling results, both dynorphin versions were chosen for further testing in the tethered format.

### Optimization of an Opioid Peptide Tethered Agonist Display

The tethered peptide strategy has been demonstrated with just a few peptides and receptors (Ishii et al. 2012; Yoshimoto et al. 2014; Nakamura et al. 2013). In developing the approach, different cell wall proteins and truncations were assayed, in addition to glycine-serine linkers. The protein 42 amino acids at the C-terminus of Flo1 ultimately led to the greatest fold change in signaling with the peptide somatostatin-14 during initial development. Flo1, thought to be involved with flocculation, has a glycosyl-phosphatidylinositol (GPI) anchor at its C-terminus (Sato et al. 2002). This anchor attaches to the plasma membrane until it is cleaved by phosphatidylinositol-specific phospholipase C (PI-PLC), when it then fixes to the inside of the yeast cell wall²⁴³ (FIG. 3).

Due to the limited application and efficacy of activating human GPCRs via this strategy, several tethering orientations for the dynorphin peptides were attempted. Anchoring with Flo42 at both the N- and C-termini of the peptides was tried. Additionally, versions with and without a glycine-serine linker between the peptide and the Flo42 region were assayed (versions with linker, FIG. 19). To enable rapid screening, the tethered peptide cassette was expressed on a plasmid under the control of a constitutive promoter. Tethered dynorphin A 1-8 and 1-13 were attempted with MOR.

Ultimately, the C-terminally tethered peptide with three glycines followed by a serine, repeated four times, led to the greatest fold change in signaling with roughly 10-fold with KOR and 17-fold with DOR in the ergosterol-producing backgrounds (FIG. 19). MOR showed slight activation with the shorter peptide in this format of roughly 2-fold in the ergosterol-producing strain, and 5-fold in the cholesterol-producing strain. The addition of a linker likely enables increased movement of the peptides, allowing greater interaction with receptor. The peptides tethered at their N-termini led to no receptor signaling, a result which correlates with literature that the N-termini of opioid peptides, and in particular the tyrosine in the first position, are integral for receptor binding and signaling (Chavkin et al. 1981).

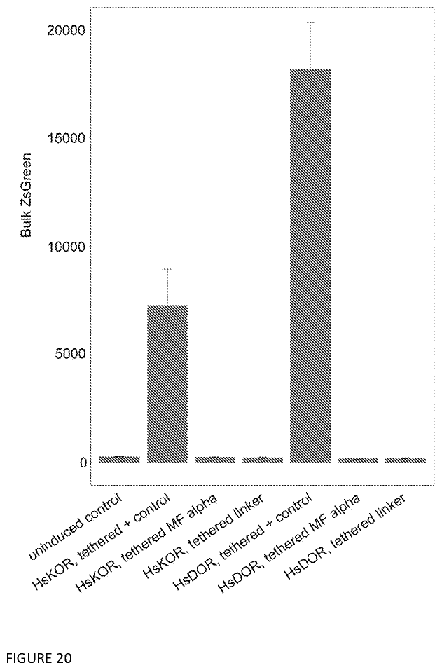

This optimized tethering orientation was then assayed in the context to be used for library peptide display. Expression was driven by a galactose-inducible promoter, pGal1, and the construct was integrated into the yeast genome. The inducible promoter was used to control the length of peptide expression and minimize potential for unwanted selection from long-term constitutive expression. Like the tethered peptide cassette, receptors were integrated into the genome to avoid downstream issues with variable plasmid copy number and sorting (Bean et al. 2022). Negative controls of the mating factor alpha peptide and the glycine-serine linker, without peptide agonist, were also expressed in this fashion, and showed no activity (FIG. 20). This strategy led to robust signaling with KOR and DOR, with fold changes of roughly 25 and 30 fold over background, respectively, in the ergosterol-producing background (FIG. 4). MOR, however, showed no signaling in either the ergosterol- or cholesterol-producing backgrounds (FIG. 4, FIG. 20). It was decided to screen MOR with the peptide libraries to determine if there were any variations that could rescue function, with the hypothesis that the sequence space assayed would likely contain variants capable of activating the receptor.

### Library Creation and Sorting

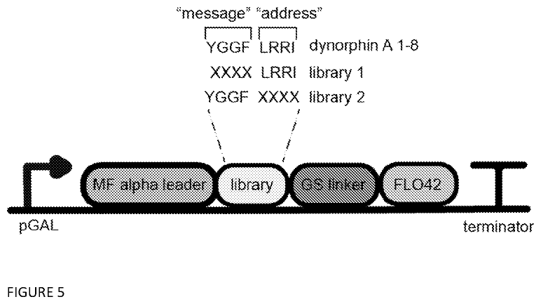

Two peptide libraries using the dynorphin A 1-8 scaffold were created. The first, henceforth referred to as library 1, site saturated the first four positions of the peptide, YGGF, using an NNK mutagenesis strategy (FIG. 5). This is canonically the “message” region, understood to be integral to opioid receptor signaling, with the tyrosine being particularly important. Some variation in this sequence has been shown to be tolerated, particularly at Gly2 and Gly3, which are thought to function primarily as a spacer. In creating this library, it was hoped that it could be determined how much variation is tolerated in this region, and how this might differ for opioid receptor sub-type.

The second library, library 2, saturated the latter four residues of the peptide, LRRI (FIG. 5). This “address” region is thought to confer receptor specificity, interacting with receptor residues further up the binding pocket (Metzger et al. 2001; Xue et al. 1994; Marie-Pepin et al. 1997), but can still be important for receptor activity. It was hypothesized that more variation would be tolerated in this region, including more sub-type specific variation.

The initial libraries were created and sequenced pre-transformation. They had 73% and 84% of theoretical amino acid complexity, respectively Table 2). The libraries were initially transformed in the cholesterol-producing yeast strains, as these showed the highest fold change and most sensitive signaling with DOR and KOR (FIG. 20). Despite protocol troubleshooting, the transformation efficiency with these strains remained low, likely due to the growth defect with this background, and was not sufficient to cover library diversity. In switching to the ergosterol-producing strains, library transformation efficiency exceeded three times the theoretical nucleotide diversity for each transformation, providing confidence that even low count library variants had a good chance of being covered.

Libraries were sorted twice on GFP fluorescence. Roughly 15 times the theoretical nucleotide complexity was sorted to ensure rare variants were assayed. A larger fraction of the library 2 variants had a GFP signal than the library 1 variants—for DOR, less than 1% of the population of library 1 had a fluorescent signal, while roughly 20% of library 2 did during the first sorts of these libraries. After the second sorts, the genomes of the positive and negative populations were prepped and the library cassettes were sequenced in technical replicate using NGS at a depth of at least 1 million reads.

### NGS Results

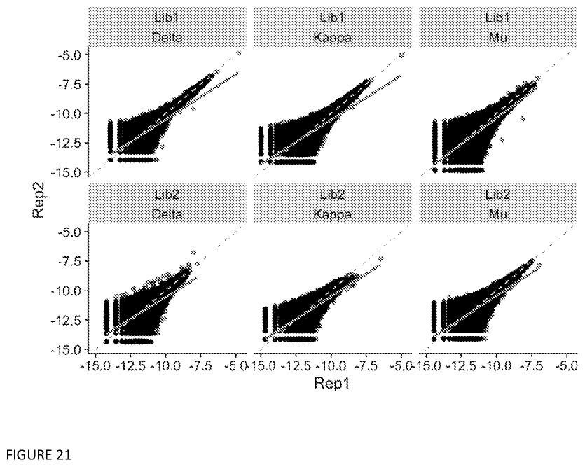

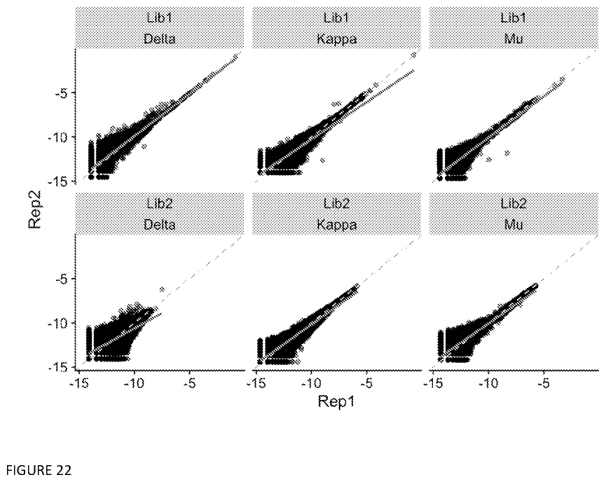

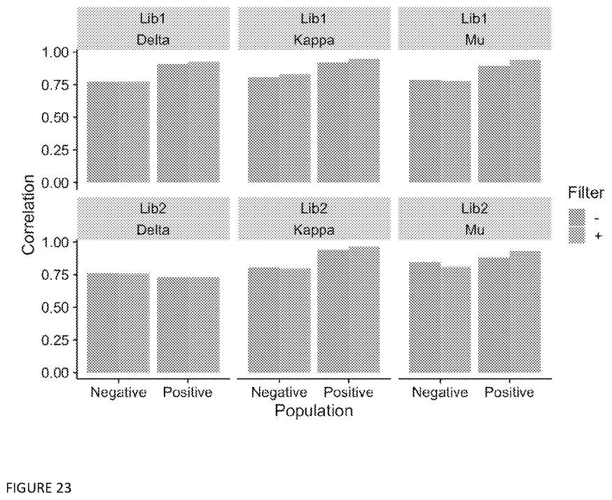

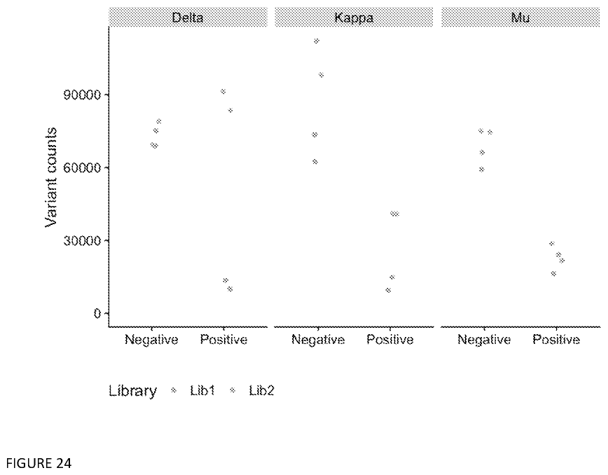

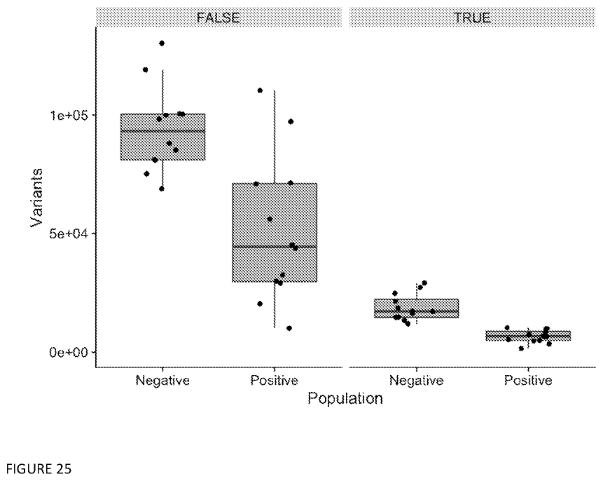

There was a high degree of correlation between the technical replicates, before and after filtering on log proportion parameters (FIGS. 21-23). There were a greater number of unique variants in the negative GFP pools compared to the positive GFP pools (FIG. 24). The exception to this was DOR, library 2, which showed the highest fraction of library members with a GFP signal. The negative pools were enriched with variants with stop codons compared to the positive pools (FIG. 25).

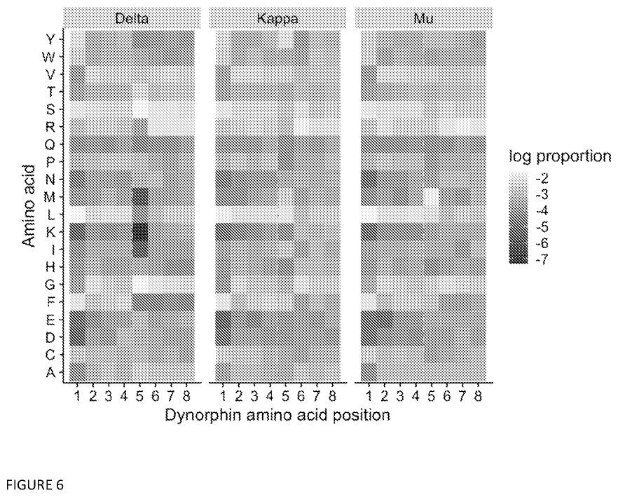

Several strategies were used to both identify unique binders for receptor sub-type and identify generally preferred residues and motifs. Log proportions between the positive and negative pools were determined with a filter of-11 (FIG. 6). While useful for highlighting potentially significant low count variants, this strategy could enrich for noise resulting from sorting errors. Because the libraries were sorted twice, and the fraction of cells with a GFP signal small, strong signalers were expected to be present in relatively high numbers in the positive pool. This expectation held true for the library 1 wild type sequences with KOR and DOR. These receptors signaled well with the wild-type peptide, and the highly conserved first four residues had the highest frequency counts in each library replicate. This motif was not as enriched by the log proportion metric (FIG. 6).

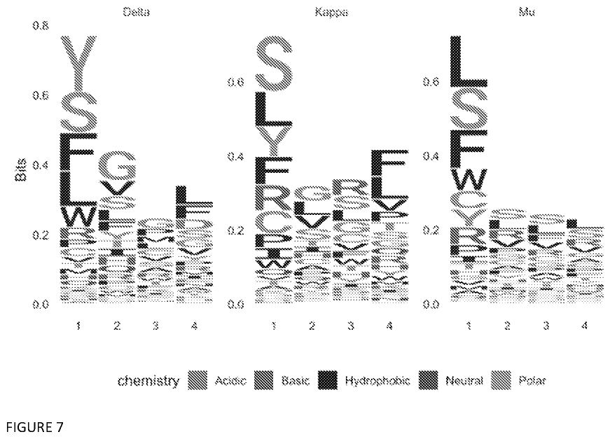

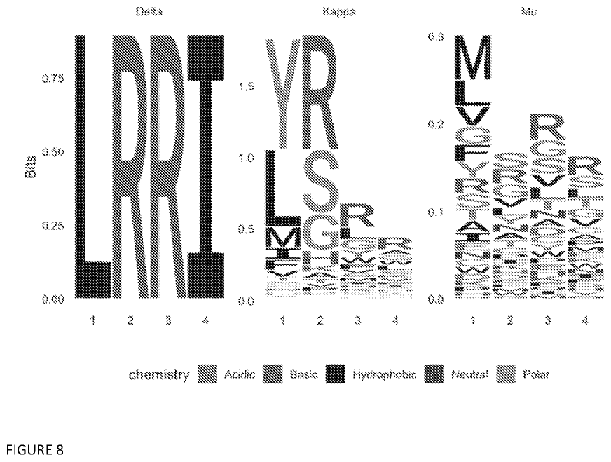

To incorporate both metrics, log proportions after a cut off of 500 nucleotide read counts were also determined (FIGS. 7 and 8).

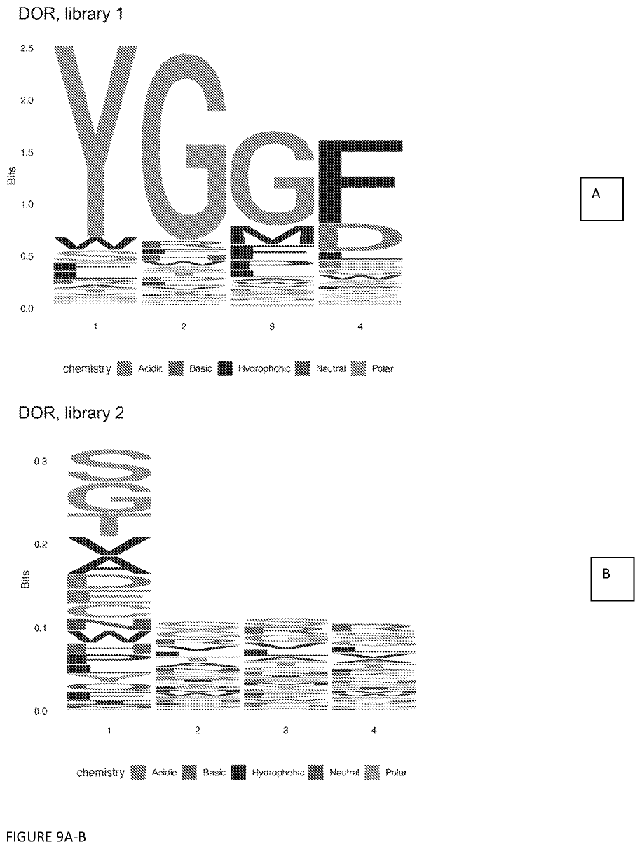

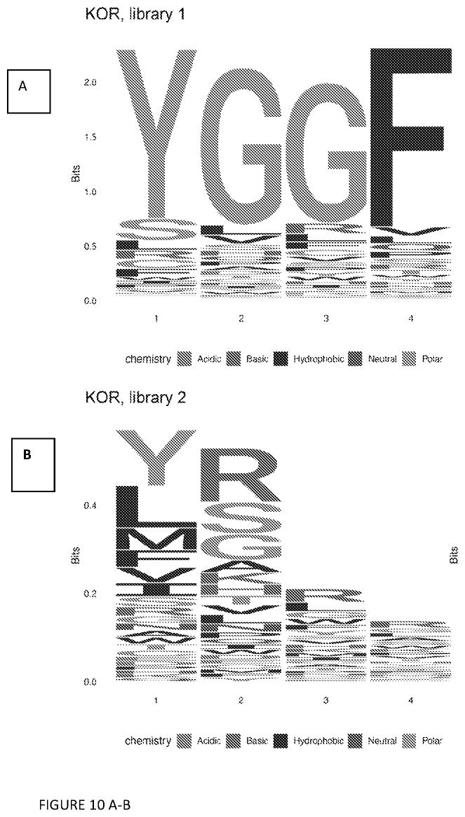

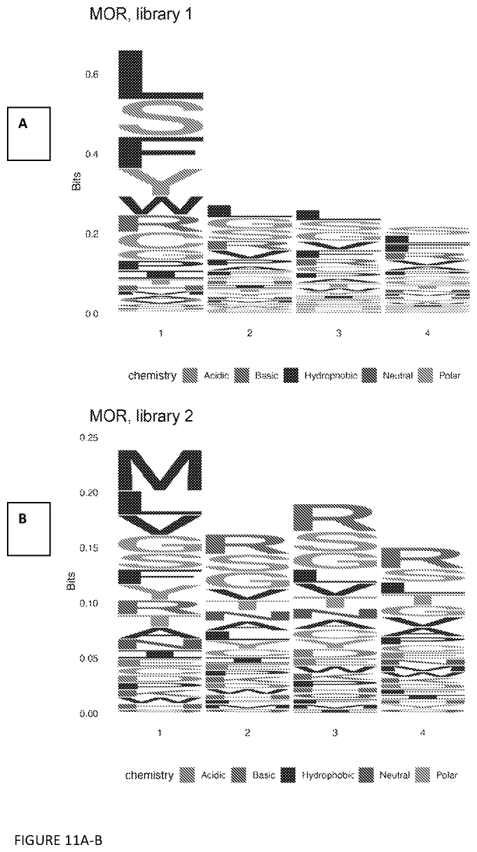

As expected, both DOR and KOR variants were predominantly the wild type peptide sequence, YGGF, in terms of total nucleotide count (FIGS. 9 and 10). There was some tolerance in this region for other amino acids, however. DOR appeared to tolerate tryptophan in place of the conserved tyrosine. These amino acids both have aromatic rings capable of hydrogen bonding. While a phenolic hydroxyl is frequently a component of opioid receptor agonists and antagonists, it's possible that the indole of tryptophan can fulfill the hydrophobic contacts and hydrogen bonding of this functional group. In contrast, serine was enriched in the first position for KOR after tyrosine (FIG. 10). Like tyrosine, this residue has a hydroxyl capable of the crucial hydrogen bonding at this region but is smaller, not having the aromatic. The solved structures of these receptors with agonists and antagonists can potentially provide insight into why certain residues are favored over others, even in the highly conserved binding pocket. Some of the conserved residues, such as V281 and H278, adopt slightly different positions in the active structures, potentially explaining why the bulkier tryptophan is selected for in DOR over the smaller serine with KOR. For MOR, leucine, serine and phenylalanine were enriched in the first position (FIG. 11).

The “address” region in library 2 is thought to confer receptor specificity by interactions primarily with extracellular loops 2 and 3, regions with high degrees of structural similarity but residue variability. Much less wild type motif was found in library 2 with DOR and KOR compared to library 1 (FIGS. 9 and 10). With all three receptors, the fifth position appeared to be particularly important for receptor activity and specificity. For DOR, serine, glycine and threonine were enriched in this fifth position. For KOR, tyrosine and hydrophobic residues, including the wild type leucine, were enriched at the fifth position by both the total positive count and the log proportion metrics. Arginine was the dominant residue in positions 6-8, which corroborates previous results that arginine at the 6th and 7th positions are important for KOR selectivity. The consensus with wild type residues at these positions makes sense because the wild type dynorphin peptide is most selective for KOR, and therefore likely less tolerant of mutations. For MOR, the hydrophobic methionine, leucine and valine were favored in the fifth position (FIG. 11).

### Determination of Unique Peptide Leads

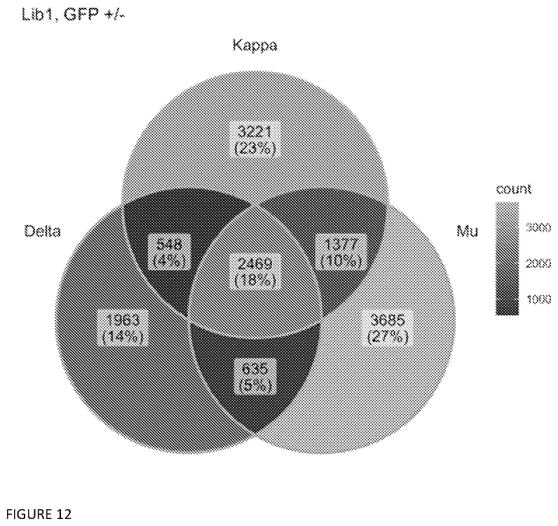

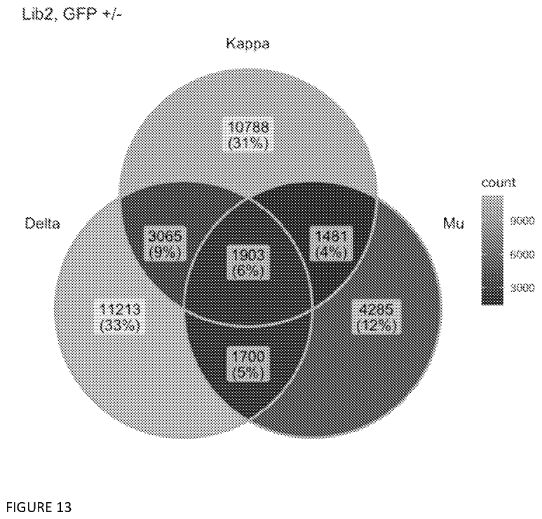

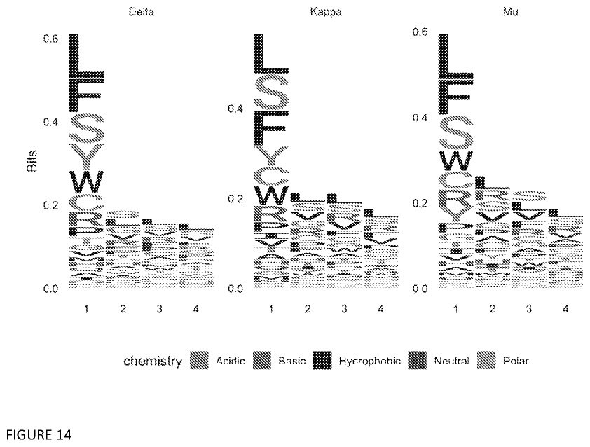

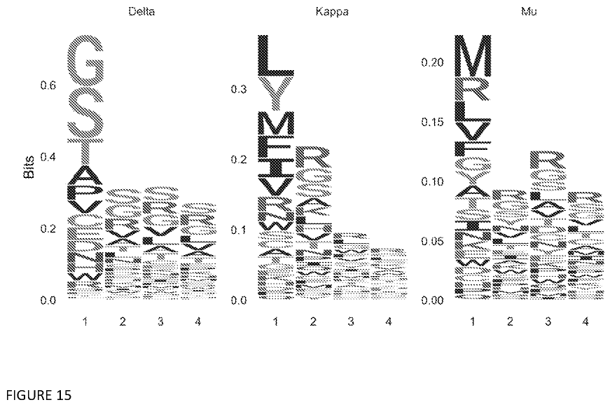

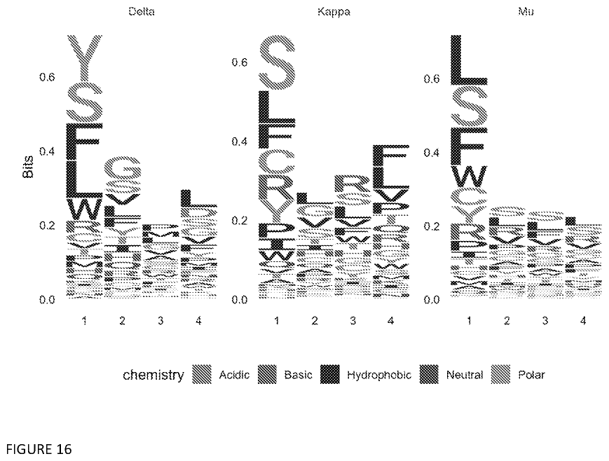

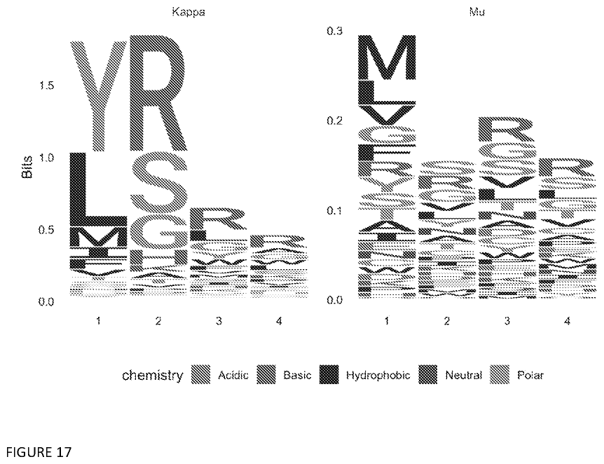

Beyond identification of general motif trends, variants unique to individual receptors were determined. For a general idea of variant specificity, plots showing receptor specificity by the log proportion metric were generated (FIGS. 12 and 13). Library 2 had a higher percentage of variants that were receptor specific, with only 6% of variants found in all receptor pools. While a greater percentage of variants meeting the log proportion cut-off in library 1 were active against all three receptors, at 18%, large numbers of variants were unique to the individual receptors (FIG. 12). Logo plots of receptor-unique variants by this metric are highlighted in FIGS. 14 and 15. To account for the noise around potentially low count variants, log proportions between positive and negative pools were determined after filtering on a minimum variant read count of 500 in the positive and negative pools. Variants that were receptor unique by this metric are highlighted in the logo plots in FIGS. 16 and 17.

Peptide candidates for further analysis were determined by manually curating the three discussed methods (Table 1). Tyrosine was enriched in position one for DOR by these metrics, and serine for KOR. Methionine was enriched in the fifth position by these metrics for MOR, and tyrosine for KOR.

### Discussion

Opioid receptor agonists are a dominant treatment for pain management despite the negative side-effects of these drugs. Current drug discovery technology is often expensive and time-consuming, both in terms of compound synthesis and use of cell culture-based assays. The work presented here provides an inexpensive way to assay hundreds of thousands of peptides for their ability to agonize human GPCRs in the fast and portable humanized yeast system. In building on previous agonist and antagonist identification studies (Bass et al. 1996; Campbell et al. 1999), this work is the first to utilize a tethered peptide strategy to identify GPCR agonists.

An optimal tethering strategy was first identified for the endogenous peptide agonist dynorphin A 1-8. Saturated libraries were then created at the “message” and “address” regions of the peptide and selected for GPCR activation at the three opioid receptor subtypes. In deep sequencing the positive and negative pools at the library regions, new agonist leads were identified. Consensus motifs for each receptor were determined based on multiple metrics, and agonist leads unique to individual receptors identified for further studies.

While there is a large body of literature detailing the effects of opioid receptor mutations on ligand function (Vardy et al. 2013; Metzger et al. 2001; Xue et al. 1994; Marie-Pepin et al. 1997; Fenalti et al. 2014; Metzger et al. 1995), there is less work that systematically details the effects of residue changes at different positions in peptide ligands. Studies that have modified peptide ligands are typically limited to single residue modifications (Lai et al. 2006; Lung et al. 1996). This work presents the first saturated mutagenesis of an opioid receptor peptide ligand, and the screening of these libraries against multiple receptors. This strategy is useful in other formats, including to screen for antagonists using an inverted reporter, and as a receptor deorphanization strategy.

### Methods

### Strains and Plasmids

Strains and plasmids are listed in Tables 4 and 5. Strains were derived from BY4741 using CRISPR-Cas9 or homology-based integration using auxotrophic markers. For CRISPR-Cas9 edits, the Yeast Toolkit was used to create Cas9 vectors as previously described (Lee et al. 2015). Yeast were transformed with this vector and appropriate repair templates, and confirmed via sequencing and colony PCR. Non-CRISPR based edits utilized parts from the Yeast Toolkit.

Non-peptide library yeast transformations were performed using the Zymo Research EZ Yeast Transformation II Kit. Peptide libraries were transformed first into Electromax DH10ß competent cells (Thermo Fisher) via electoporation after plasmid assembly. Transformed cultures were grown overnight and then mini-prepped. Transformation efficiency was determined via serial dilution plating after one day. Plasmid was digested via NotI-HF (NEB) to linearize the vector for genomic integration in yeast. After digestion, DNA was purified using a Zymo Clean and Concentrator kit. Roughly 75 μg of DNA was transformed into yeast in iterative transformations for each library using a protocol for large scale yeast transformations previously described (Benatuil et al. 2010). Serial dilution plating was used to determine transformation efficiency after 2-3 days.

Plasmids were constructed using Golden Gate assembly of components from the Yeast Toolkit with some adaptations. Receptors were expressed on either 2μ URA3 backbones driven by the CCW12 promoter or integrated into the yeast genome using a URA3 auxotrophic marker and driven by the TDH3 promoter. Tethered peptide cassettes were expressed on either a Cen6 HIS3 backbone or integrated into the yeast genome using HIS3 auxotrophic markers and were driven by pHHF2 for plasmid-based expression, and pGal1 for integrated expression. Oligos were ordered as gblocks or single stranded DNA from IDT.

### Functional Assays

For non-sorting flow cytometry assays, yeast colonies were picked and grown overnight to saturation in SD-URA or SD-URA/His media at pH 5.8 in a 2.2 mL deep well plate or 15 mL tubes in either a plate-shaking incubator (30° C., 1000 rpm, 3 mm orbital) or a floor shaking incubator (30° C., 300 rpm). Cultures were diluted 1:25 the next day in either SD-URA or SD-URA/His media at pH 7.1 buffered with 100 mM MOPS. When appropriate, ligands were added, the cultures were grown for 8 hours, and spun down and resuspended in 150 uL TE pH 8.0. Flow cytometry was performed using a Sony SA3800 spectral analyzer. 10,000 events were assayed while filtering for singlet populations.

For cell sorting experiments, post-transformation outgrowths were grown overnight in SD-URA/His pH 5.8 media, and cultures were passaged the following day 1:25 to minimize dead and untransformed cells. The following day the saturated cultures were washed three times in SD-URA/His pH 7.1 100 mM MOPS media without a carbon source to minimize glucose carry-over. Cultures were then diluted 1:10 their initial volume in SD-URA/His pH. 7.1 100 mM MOPS 2% galactose 0.2% glucose media and grown for 24 hours. Cells were then resuspended in an equal volume of TE pH 8.0 and 14-15 million events were sorted using a SH800S Cell Sorter (Sony) on GFP fluorescence, gated for singlets. Cells were sorted into SD-His/URA pH 5.8 media with pen/strep and grown for two days post sorting. Cultures were again washed and induced with galactose and sorted via the same parameters into positive and negative pools and grown up for 2 days. Genome extractions were performed using a Zymo Yeast Star kit. Primers that annealed roughly 75 nucleotides upstream and downstream of the peptide-encoding library were used to PCR the variable region for DNA amplicon next generation sequencing. Approximately 60 ng of genomic DNA was used for PCRs.

### Next Generation Sequencing, Alignment and Analysis

DNA amplicons were sequenced using a MiSeq V2, PE150 in technical replicate. Each of the 24 samples was aligned with bwa (version 0.7.17-r1188) (Li et al. 2009) using the mem algorithm, against a “short genome” index build from the plasmid vector sequence containing the wild type 8-AA sequence. Alignment options were adjusted to minimize indel calls (-B 0-O 12, 12-L 4,4-T 25-k 17). All resulting alignments had less than 6% indels reported (I or D in the alignment CIGAR string).

Alignment records were filtered using samtools (version 1.7) (Danecek et al. 2021) to retain only mapped, properly paired, plus strand reads, without indels, that overlapped the 8-AA/24-nucleotide (“NT”) target region. Combined with knowledge of the plasmid structure, this allowed the isolation of each of the two (Area1, Area2) 4 AA/12 NT sequences.

Quantification of the unique 12-NT sequences for each Area (i.e., the first 12 NTs for Area1 libraries and the second 12 NTs for Area2 libraries) was performed in R (version 4.0.3). Quantification of the unique 4-AA sequences for each Area was obtained by translating NTs to AAs using the standard genetic code.

Unique NT and AA counts were first recorded for individual TRs. These raw counts were then combined into per-OR-Area-Pos-or-Neg, per-OR-Area and per-Area matrices along with sequencing-depth-normalized counts (to 1 million reads per library). The effect of selection pressure on the observed AA motifs was also captured, as 1) the difference between the number of normalized NT sequences for each unique AA motif in the Pos experiment and the corresponding number in the Neg experiment; and 2) Fold change, and Log2 Fold change of Pos/Neg normalized count ratios (after replacing 0 counts with 0.5).

### REFERENCES

1. Lengger, B. & Jensen, M. K. Engineering G protein-coupled receptor signalling in yeast for biotechnological and medical purposes. FEMS Yeast Res. 20, foz087 (2019).
2. Zhou, Q. et al. Common activation mechanism of class A GPCRs. eLife 8, e50279 (2019).
3. Hauser, A. S., Attwood, M. M., Rask-Andersen, M., Schioth, H. B. & Gloriam, D. E. Trends in GPCR drug discovery: new agents, targets and indications. Nat. Rev. Drug Discov. 16, 829-842 (2017).
4. Hauser, A. S., Attwood, M. M., Rask-Andersen, M., Schioth, H. B. & Gloriam, D. E. Trends in GPCR drug discovery: new agents, targets and indications. Nat. Rev. Drug Discov. 16, 829-842 (2017).
5. Raehal, K. M., Walker, J. K. L. & Bohn, L. M. Morphine side effects in beta-arrestin 2 knockout mice. J. Pharmacol. Exp. Ther. 314, 1195-1201 (2005).
6. Hughes, J., Rees, S., Kalindjian, S. & Philpott, K. Principles of early drug discovery. Br. J. Pharmacol. 162, 1239-1249 (2011).
7. Velloso, L. A. et al. Cross-talk between the insulin and angiotensin signaling systems. Proc. Natl. Acad. Sci. U.S.A 93, 12490-12495 (1996).
8. Prezeau, L. et al. Functional crosstalk between GPCRs: with or without oligomerization. Curr. Opin. Pharmacol. 10, 6-13 (2010).
9. Thomsen, W., Frazer, J. & Unett, D. Functional assays for screening GPCR targets. Curr. Opin. Biotechnol. 16, 655-665 (2005).
10. Kachroo, A. H., Vandeloo, M., Greco, B. M. & Abdullah, M. Humanized yeast to model human biology, disease and evolution. Dis. Model. Mech. 15, dmm049309 (2022).
11. Kachroo, A. H. et al. Systematic humanization of yeast genes reveals conserved functions and genetic modularity. Science 348, 921-925 (2015).
12. Versele, M., Lemaire, K. & Thevelein, J. M. Sex and sugar in yeast: two distinct GPCR systems. EMBO Rep. 2, 574-579 (2001).
13. Weis, W. I. & Kobilka, B. K. The Molecular Basis of G Protein-Coupled Receptor Activation. Annu. Rev. Biochem. 87, 897-919 (2018).
14. Hurowitz, E. H. et al. Genomic Characterization of the Human Heterotrimeric G Protein α, β, and γ Subunit Genes. DNA Res. 7, 111-120 (2000).
15. Okashah, N. et al. Variable G protein determinants of GPCR coupling selectivity. Proc. Natl. Acad. Sci. 116, 12054-12059 (2019).
16. Apanovitch, D. M., Slep, K. C., Sigler, P. B. & Dohlman, H. G. Sst2 Is a GTPase-Activating Protein for Gpa1: Purification and Characterization of a Cognate RGS-Ga Protein Pair in Yeast. Biochemistry 37, 4815-4822 (1998).
17. Alvaro, C. G. & Thorner, J. Heterotrimeric G Protein-coupled Receptor Signaling in Yeast Mating Pheromone Response. J. Biol. Chem. 291, 7788-7795 (2016).
18. Chen, R. E. & Thorner, J. Function and regulation in MAPK signaling pathways: Lessons learned from the yeast Saccharomyces cerevisiae. Biochim. Biophys. Acta BBA-Mol. Cell Res. 1773, 1311-1340 (2007).
19. Brown, A. et al. Functional coupling of mammalian receptors to the yeast mating pathway using novel yeast/mammalian G protein alpha-subunit chimeras. Yeast 16, 11-22 (2000).
20. Mukherjee, K., Bhattacharyya, S. & Peralta-Yahya, P. GPCR-Based Chemical Biosensors for Medium-Chain Fatty Acids. ACS Synth. Biol. 4, 1261-1269 (2015).
21. Erlenbach, I. et al. Functional expression of M (1), M (3) and M (5) muscarinic acetylcholine receptors in yeast. J. Neurochem. 77, 1327-1337 (2001).
22. Shaw, W. M. et al. Screening microbially produced 49-tetrahydrocannabinol using a yeast biosensor workflow. Nat. Commun. 13, 5509 (2022).
23. Lengger, B. et al. Serotonin G Protein-Coupled Receptor-Based Biosensing Modalities in Yeast. ACS Sens. 7, 1323-1335 (2022).
24. Ladds, G., Goddard, A. & Davey, J. Functional analysis of heterologous GPCR signalling pathways in yeast. Cell Trends Biotechnol. 23, 367-373 (2005).
25. Armbruster, B. N., Li, X., Pausch, M. H., Herlitze, S. & Roth, B. L. Evolving the lock to fit the key to create a family of G protein-coupled receptors potently activated by an inert ligand. Proc. Natl. Acad. Sci. U.S.A 104, 5163-5168 (2007).

26 Dong, S., Rogan, S. C. & Roth, B. L. Directed molecular evolution of DREADDs: a generic approach to creating next-generation RASSLs. Nat. Protoc. 5, 561-573 (2010).
27. King, K., Dohlman, H. G., Thorner, J., Caron, M. G. & Lefkowitz, R. J. Control of yeast mating signal transduction by a mammalian beta 2-adrenergic receptor and Gs alpha subunit. Science 250, 121-123 (1990).
28. Price, L. A., Kajkowski, E. M., Hadcock, J. R., Ozenberger, B. A. & Pausch, M. H. Functional coupling of a mammalian somatostatin receptor to the yeast pheromone response pathway. Mol. Cell. Biol. 15, 6188-6195 (1995).
29. Klein, C. et al. Identification of surrogate agonists for the human FPRL-1 receptor by autocrine selection in yeast. Nat. Biotechnol. 16, 1334-1337 (1998).
30. Yasi, E. A. et al. Rapid Deorphanization of Human Olfactory Receptors in Yeast. Biochemistry 58, 2160-2166 (2019).
31. Lu, M., Echeverri, F. & Moyer, B. D. Endoplasmic reticulum retention, degradation, and aggregation of olfactory G-protein coupled receptors. Traffic Cph. Den. 4, 416-433 (2003).
32. Erlenbach, I. et al. Single amino acid substitutions and deletions that alter the G protein coupling properties of the V2 vasopressin receptor identified in yeast by receptor random mutagenesis. J. Biol. Chem. 276, 29382-29392 (2001).
33. O'Malley, M. A. et al. Progress toward heterologous expression of active G-protein-coupled receptors in Saccharomyces cerevisiae: Linking cellular stress response with translocation and trafficking. Protein Sci. Publ. Protein Soc. 18, 2356-2370 (2009).
34. Weston, C. et al. Modulation of Glucagon Receptor Pharmacology by Receptor Activity-modifying Protein-2 (RAMP2). J. Biol. Chem. 290, 23009-23022 (2015).
35. Bean, B. D. M. et al. Functional expression of opioid receptors and other human GPCRs in yeast engineered to produce human sterols. Nat. Commun. 13, 2882 (2022).
36. Uddin, M. S., Hauser, M., Naider, F. & Becker, J. M. The N-terminus of the yeast G protein-coupled receptor Ste2p plays critical roles in surface expression, signaling, and negative regulation. Biochim. Biophys. Acta 1858, 715-724 (2016).
37. Iguchi, Y. et al. Control of signalling properties of human somatostatin receptor subtype-5 by additional signal sequences on its amino-terminus in yeast. J. Biochem. (Tokyo) 147, 875-884 (2010).
38. Thomas, C. J. et al. Uncoupling conformational change from GTP hydrolysis in a heterotrimeric G protein alpha-subunit. Proc. Natl. Acad. Sci. U.S.A 101, 7560-7565 (2004).
39. Hanlon, C. D. & Andrew, D. J. Outside-in signaling—a brief review of GPCR signaling with a focus on the Drosophila GPCR family. J Cell Sci 128, 3533-3542 (2015).
40. Jean-Charles, P.-Y., Kaur, S. & Shenoy, S. K. GPCR signaling via β-arrestin-dependent mechanisms. J. Cardiovasc. Pharmacol. 70, 142-158 (2017).
41. Tang, X., Wang, Y., Li, D., Luo, J. & Liu, M. Orphan G protein-coupled receptors (GPCRs): biological functions and potential drug targets. Acta Pharmacol. Sin. 33, 363-371 (2012).
42. Yu, R. C. et al. The Alpha Project, a model system for systems biology research. IET Syst. Biol. 2, 222-233 (2008).
43. Brent, R. Cell signaling: What is the signal and what information does it carry? FEBS Lett. 583, 4019-4024 (2009).
44. Shaw, W. M. et al. Engineering a Model Cell for Rational Tuning of GPCR Signaling. Cell 177, 782-796.e27 (2019).
45. Jones, E. M. et al. A Scalable, Multiplexed Assay for Decoding GPCR-Ligand Interactions with RNA Sequencing. Cell Syst. 8, 254-260.e6 (2019).
46. Chen, H. et al. A Forward Chemical Genetic Screen Reveals Gut Microbiota Metabolites That Modulate Host Physiology. Cell 177, 1217-1231.e18 (2019).
47. Roth, B. L. How structure informs and transforms chemogenetics. Curr. Opin. Struct. Biol. 57, 9-16 (2019).
48. Kenakin, T. & Christopoulos, A. Signalling bias in new drug discovery: detection, quantification and therapeutic impact. Nat. Rev. Drug Discov. 12, 205 (2012).
49. Liu, R., Wong, W. & Ijerman, A. P. Human G protein-coupled receptor studies in Saccharomyces cerevisiae. Biochem. Pharmacol. 114, 103-115 (2016).
50. Strader, C. D. et al. Allele-specific activation of genetically engineered receptors. J. Biol. Chem. 266, 5-8 (1991).
51. Conklin, B. R. et al. Engineering GPCR signaling pathways with RASSLs. Nat. Methods 5, 673-678 (2008).
52. Coward, P. et al. Controlling signaling with a specifically designed Gi-coupled receptor. Proc. Natl. Acad. Sci. U.S.A 95, 352-357 (1998).
53. Hsiao, E. C. et al. Osteoblast expression of an engineered Gs-coupled receptor dramatically increases bone mass. Proc. Natl. Acad. Sci. U.S.A 105, 1209-1214 (2008).
54. Roth, B. L. DREADDs for Neuroscientists. Neuron 89, 683-694 (2016).
55. Alexander, G. M. et al. Remote control of neuronal activity in transgenic mice expressing evolved G protein-coupled receptors. Neuron 63, 27-39 (2009).
56. Zhu, H. et al. Chemogenetic Inactivation of Ventral Hippocampal Glutamatergic Neurons Disrupts Consolidation of Contextual Fear Memory. Neuropsychopharmacology 39, 1880-1892 (2014).
57. Chavkin, C. et al. Salvinorin A, an Active Component of the Hallucinogenic Sage Salvia divinorum Is a Highly Efficacious K-Opioid Receptor Agonist: Structural and Functional Considerations. J. Pharmacol. Exp. Ther. 308, 1197-1203 (2004).
58. Billups, D., Billups, B., Challiss, R. A. J. & Nahorski, S. R. Modulation of Gq protein-coupled IP3 and Ca2+ signaling by the membrane potential. J. Neurosci. Off. J. Soc. Neurosci. 26, 9983-9995 (2006).
59. Wettschureck, N. & Offermanns, S. Mammalian G Proteins and Their Cell Type Specific Functions. Physiol. Rev. 85, 1159-1204 (2005).
60. Vardy, E. et al. A New DREADD Facilitates the Multiplexed Chemogenetic Interrogation of Behavior. Neuron 86, 936-946 (2015).
61. Guettier, J.-M. et al. A chemical-genetic approach to study G protein regulation of β cell function in vivo. Proc. Natl. Acad. Sci. 106, 19197-19202 (2009).
62. Nakajima, K. & Wess, J. Design and Functional Characterization of a Novel, Arrestin-Biased Designer G Protein-Coupled Receptor. Mol. Pharmacol. 82, 575-582 (2012).
63. Hudson, B. D. et al. Chemically engineering ligand selectivity at the free fatty acid receptor 2 based on pharmacological variation between species orthologs. FASEB J. 26, 4951-4965 (2012).
64. Mahler, S. V. & Aston-Jones, G. CNO Evil? Considerations for the Use of DREADDs in Behavioral Neuroscience. Neuropsychopharmacology 43, 934-936 (2018).
65. Kroeze, W. K. et al. PRESTO-TANGO: an open-source resource for interrogation of the druggable human GPCR-ome. Nat. Struct. Mol. Biol. 22, 362-369 (2015).
66. Peisajovich, S. G., Garbarino, J. E., Wei, P. & Lim, W. A. Rapid Diversification of Cell Signaling Phenotypes by Modular Domain Recombination. Science 328, 368-372 (2010).
67. Packer, M. S. & Liu, D. R. Methods for the directed evolution of proteins. Nat. Rev. Genet. 16, 379-394 (2015).
68. Benatuil, L., Perez, J. M., Belk, J. & Hsieh, C.-M. An improved yeast transformation method for the generation of very large human antibody libraries. Protein Eng. Des. Sel. 23, 155-159 (2010).
69. Huang, Y. & Thathiah, A. Regulation of neuronal communication by G protein-coupled receptors. FEBS Lett. 589, 1607-1619 (2015).
70. Cordomí, A., Fourmy, D. & Tikhonova, I. G. Gut hormone GPCRs: structure, function, drug discovery. Curr. Opin. Pharmacol. 31, 63-67 (2016).
71. Patriarchi, T. et al. Ultrafast neuronal imaging of dopamine dynamics with designed genetically encoded sensors. Science 360, (2018).
72. Billerbeck, S. et al. A scalable peptide-GPCR language for engineering multicellular communication. Nat. Commun. 9, 5057 (2018).
73. Pierce, K. L., Premont, R. T. & Lefkowitz, R. J. Seven-transmembrane receptors. Nat. Rev. Mol. Cell Biol. 3, 639-650 (2002).
74. Elion, E. A. Pheromone response, mating and cell biology. Curr. Opin. Microbiol. 3, 573-581 (2000).
75. Sarramegna, V., Talmont, F., Demange, P. & Milon, A. Heterologous expression of G-protein-coupled receptors: comparison of expression systems from the standpoint of large-scale production and purification. Cell. Mol. Life Sci. CMLS 60, 1529-1546 (2003).
76. Lagane, B. et al. Role of Sterols in Modulating the Human μ-Opioid Receptor Function in Saccharomyces cerevisiae. J. Biol. Chem. 275, 33197-33200 (2000).
77. O'Malley, M. A. et al. Progress toward heterologous expression of active G-protein-coupled receptors in Saccharomyces cerevisiae: Linking cellular stress response with translocation and trafficking. Protein Sci. Publ. Protein Soc. 18, 2356-2370 (2009).
78. Butz, J. A., Niebauer, R. T. & Robinson, A. S. Co-expression of molecular chaperones does not improve the heterologous expression of mammalian G-protein coupled receptor expression in yeast. Biotechnol. Bioeng. 84, 292-304 (2003).
79. Yoo, J. I. & O'Malley, M. A. Tuning Vector Stability and Integration Frequency Elevates Functional GPCR Production and Homogeneity in Saccharomyces cerevisiae. ACS Synth. Biol. 7, 1763-1772 (2018).
80. Schütz, M. et al. Directed evolution of G protein-coupled receptors in yeast for higher functional production in eukaryotic expression hosts. Sci. Rep. 6, 1-16 (2016).
81. Ritter, S. L. & Hall, R. A. Fine-tuning of GPCR activity by receptor-interacting proteins. Nat. Rev. Mol. Cell Biol. 10, 819-830 (2009).
82. Chan, H. C. S. et al. Enhancing the Signaling of GPCRs via Orthosteric Ions. ACS Cent. Sci. 6, 274-282 (2020).
83. Opekarová, M. & Tanner, W. Specific lipid requirements of membrane proteins—a putative bottleneck in heterologous expression. Biochim. Biophys. Acta 1610, 11-22 (2003).
84. Paila, Y. D. & Chattopadhyay, A. Membrane cholesterol in the function and organization of G-protein coupled receptors. Subcell. Biochem. 51, 439-466 (2010).
85. Genheden, S., Essex, J. W. & Lee, A. G. G protein coupled receptor interactions with cholesterol deep in the membrane. Biochim. Biophys. Acta BBA-Biomembr. 1859, 268-281 (2017).
86. Guixà-González, R. et al. Membrane cholesterol access into a G-protein-coupled receptor. Nat. Commun. 8, 1-12 (2017).
87. Taghon, G. J., Rowe, J. B., Kapolka, N. J. & Isom, D. G. Predictable cholesterol binding sites in GPCRs lack consensus motifs. Struct. Lond. Engl. 1993 29, 499-506.e3 (2021).
88. Morioka, S. et al. Effect of sterol composition on the activity of the yeast G-protein-coupled receptor Ste2. Appl. Microbiol. Biotechnol. 97, 4013-4020 (2013).
89. Souza, C. M. et al. A stable yeast strain efficiently producing cholesterol instead of ergosterol is functional for tryptophan uptake, but not weak organic acid resistance. Metab. Eng. 13, 555-569 (2011).
90. Waldhoer, M., Bartlett, S. E. & Whistler, J. L. Opioid Receptors. Annu. Rev. Biochem. 73, 953-990 (2004).
91. Skolnick, P. The Opioid Epidemic: Crisis and Solutions. Annu. Rev. Pharmacol. Toxicol. 58, 143-159 (2018).
92. Olesnicky, N. S., Brown, A. J., Dowell, S. J. & Casselton, L. A. A constitutively active G-protein-coupled receptor causes mating self-compatibility in the mushroom Coprinus. EMBO J. 18, 2756-2763 (1999).
93. Erdman, S., Lin, L., Malczynski, M. & Snyder, M. Pheromone-regulated Genes Required for Yeast Mating Differentiation. J. Cell Biol. 140, 461-483 (1998).
94. Heiman, M. G. & Walter, P. Prmlp, a Pheromone-Regulated Multispanning Membrane Protein, Facilitates Plasma Membrane Fusion during Yeast Mating. J. Cell Biol. 151, 719-730 (2000).
95. Erickson, J. R. et al. Edg-2/Vzg-1 couples to the yeast pheromone response pathway selectively in response to lysophosphatidic acid. J. Biol. Chem. 273, 1506-1510 (1998).
96. Casey, J. R., Grinstein, S. & Orlowski, J. Sensors and regulators of intracellular pH. Nat. Rev. Mol. Cell Biol. 11, 50-61 (2010).
97. Harris, I., Jones, E. W. & Aldred, C. N. The pH and Lactic Acid Content of the Cerebrospinal Fluid. J. Neurol. Psychopathol. 15, 46-52 (1934).
98. Edgar, R. C. MUSCLE: multiple sequence alignment with high accuracy and high throughput. Nucleic Acids Res. 32, 1792-1797 (2004).
99. Stevens, C. W. The evolution of vertebrate opioid receptors. Front. Biosci. J. Virtual Libr. 14, 1247-1269 (2009).
100. Raynor, K. et al. Pharmacological characterization of the cloned kappa-, delta-, and mu-opioid receptors. Mol. Pharmacol. 45, 330-334 (1994).
101. Toll, L. et al. Standard binding and functional assays related to medications development division testing for potential cocaine and opiate narcotic treatment medications. NIDA Res. Monogr. 178, 440-466 (1998).
102. Nickolls, S. A., Waterfield, A., Williams, R. E. & Kinloch, R. A. Understanding the effect of different assay formats on agonist parameters: a study using the μ-opioid receptor. J. Biomol. Screen. 16, 706-716 (2011).
103. Kreil, G. et al. Deltorphin, a novel amphibian skin peptide with high selectivity and affinity for delta opioid receptors. Eur. J. Pharmacol. 162, 123-128 (1989).
104. Bilsky, E. J. et al. SNC 80, a selective, nonpeptidic and systemically active opioid delta agonist. J. Pharmacol. Exp. Ther. 273, 359-366 (1995).
105. Camilleri, M. Asimadoline, a k-Opioid Agonist, and Visceral Sensation. Neurogastroenterol. Motil. Off. J. Eur. Gastrointest. Motil. Soc. 20, 971-979 (2008).
106. Yasuda, K. et al. Cloning and functional comparison of kappa and delta opioid receptors from mouse brain. Proc. Natl. Acad. Sci. U.S.A 90, 6736-6740 (1993).
107. Burford, N. T. et al. Identification of Selective Agonists and Positive Allosteric Modulators for μ- and δ-Opioid Receptors from a Single High-Throughput Screen. SLAS Discov. 19, 1255-1265 (2014).
108. Tonini, M. et al. Endomorphin-1 and endomorphin-2 activate mu-opioid receptors in myenteric neurons of the guinea-pig small intestine. Naunyn. Schmiedebergs Arch. Pharmacol. 358, 686-689 (1998).
109. LaVigne, J., Keresztes, A., Chiem, D. & Streicher, J. M. The endomorphin-1/2 and dynorphin-B peptides display biased agonism at the mu opioid receptor. Pharmacol. Rep. 72, 465-471 (2020).
110. Broad, J. et al. Human native kappa opioid receptor functions not predicted by recombinant receptors: Implications for drug design. Sci. Rep. 6, 30797 (2016).
111. Malatynska, E. et al. Human delta opioid receptor: functional studies on stably transfected Chinese hamster ovary cells after acute and chronic treatment with the selective nonpeptidic agonist SNC-80. J. Pharmacol. Exp. Ther. 278, 1083-1089 (1996).
112. Knapp, R. J. et al. Structure-activity relationships for SNC80 and related compounds at cloned human delta and mu opioid receptors. J. Pharmacol. Exp. Ther. 277, 1284-1291 (1996).
113. Quitterer, U., Pohl, A., Langer, A., Koller, S. & Abdalla, S. A cleavable signal peptide enhances cell surface delivery and heterodimerization of Cerulean-tagged angiotensin II AT1 and bradykinin B2 receptor. Biochem. Biophys. Res. Commun. 409, 544-549 (2011).
114. Martoglio, B. & Dobberstein, B. Signal sequences: more than just greasy peptides. Trends Cell Biol. 8, 410-415 (1998).
115. Rutz, C., Klein, W. & Schülein, R. N-Terminal Signal Peptides of G Protein-Coupled Receptors: Significance for Receptor Biosynthesis, Trafficking, and Signal Transduction. Prog. Mol. Biol. Transl. Sci. 132, 267-287 (2015).
116. Fitzgerald, I. & Glick, B. S. Secretion of a foreign protein from budding yeasts is enhanced by cotranslational translocation and by suppression of vacuolar targeting. Microb. Cell Factories 13, 125 (2014).
117. Mayer, P. & Hö11t, V. Allelic and somatic variations in the endogenous opioid system of humans. Pharmacol. Ther. 91, 167-177 (2001).
118. Olsen, T. C. R., Rasmussen, A. R., Kringen, M. K. & Molden, E. A girl of early school-age with no response to opioids during general anaesthesia. Tidsskr. Den Nor. Legeforening (2019) doi: 10.4045/tidsskr. 18.0561.
119. Ravindranathan, A. et al. Functional characterization of human variants of the mu-opioid receptor gene. Proc. Natl. Acad. Sci. U.S.A 106, 10811-10816 (2009).
120. Lötsch, J. & Geisslinger, G. Are mu-opioid receptor polymorphisms important for clinical opioid therapy? Trends Mol. Med. 11, 82-89 (2005).
121. Duarte, M. L. & Devi, L. A. Post-translational modifications of Opioid Receptors. Trends Neurosci. 43, 417-432 (2020).
122. Shiwarski, D. J., Crilly, S. E., Dates, A. & Puthenveedu, M. A. Dual RXR motifs regulate nerve growth factor-mediated intracellular retention of the delta opioid receptor. Mol. Biol. Cell 30, 680-690 (2019).
123. Geva, Y. & Schuldiner, M. The back and forth of cargo exit from the endoplasmic reticulum. Curr. Biol. CB 24, R130-136 (2014).
124. Li, J.-G., Chen, C. & Liu-Chen, L.-Y. N-Glycosylation of the Human K Opioid Receptor Enhances Its Stability but Slows Its Trafficking along the Biosynthesis Pathway. Biochemistry 46, 10960-10970 (2007).
125. Lackman, J. J., Markkanen, P. M. H., Hogue, M., Bouvier, M. & Petäjä-Repo, U. E. N-Glycan-dependent and-independent Quality Control of Human 8 Opioid Receptor N-terminal Variants*. J. Biol. Chem. 289, 17830-17842 (2014).
126. Markkanen, P. M. H. & Petäjä-Repo, U. E. N-glycan-mediated quality control in the endoplasmic reticulum is required for the expression of correctly folded delta-opioid receptors at the cell surface. J. Biol. Chem. 283, 29086-29098 (2008).
127. Chaturvedi, K., Shahrestanifar, M. & Howells, R. D. mu Opioid receptor: role for the amino terminus as a determinant of ligand binding affinity. Brain Res. Mol. Brain Res. 76, 64-72 (2000).
128. Deng, H. B. et al. Role for the C-terminus in agonist-induced mu opioid receptor phosphorylation and desensitization. Biochemistry 39, 5492-5499 (2000).
129. Manglik, A. et al. Crystal structure of the μ-opioid receptor bound to a morphinan antagonist. Nature 485, 321-326 (2012).
130. Feng, G.-J. et al. Selective Interactions between Helix VIII of the Human μ-Opioid Receptors and the C Terminus of Periplakin Disrupt G Protein Activation*. J. Biol. Chem. 278, 33400-33407 (2003).
131. Platt, F. M. et al. Disorders of cholesterol metabolism and their unanticipated convergent mechanisms of disease. Annu. Rev. Genomics Hum. Genet. 15, 173-194 (2014).
132. Cook, R. P. CHAPTER 4-DISTRIBUTION OF STEROLS IN ORGANISMS AND IN TISSUES. in (1958). doi: 10.1016/B978-1-4832-2772-6.50009-7.
133. Ehrenworth, A. M., Claiborne, T. & Peralta-Yahya, P. Medium-throughput screen of microbially produced serotonin via a GPCR-based sensor. Biochemistry 56, 5471-5475 (2017).
134. Weston, C., Poyner, D., Patel, V., Dowell, S. & Ladds, G. Investigating G protein signalling bias at the glucagon-like peptide-1 receptor in yeast. Br. J. Pharmacol. 171, 3651-3665 (2014).
135. Ishii, J. & Kondo, A. Cell Wall Trapping of Autocrine Peptides for Human G-Protein-Coupled Receptors on the Yeast Cell Surface. PLOS One 7, (2012).
136. Crocetti, L. et al. Synthesis and Pharmacological Evaluation of New Pyridazin-Based Thioderivatives as Formyl Peptide Receptor (FPR) Agonists. Drug Dev. Res. 74, 259-271 (2013).
137. Grant, M., Alturaihi, H., Jaquet, P., Collier, B. & Kumar, U. Cell growth inhibition and functioning of human somatostatin receptor type 2 are modulated by receptor heterodimerization. Mol. Endocrinol. Baltim. Md 22, 2278-2292 (2008).
138. Pindon, A. et al. Differences in signal transduction of two 5-HT4 receptor splice variants: compound specificity and dual coupling with Galphas- and Galphai/o-proteins. Mol. Pharmacol. 61, 85-96 (2002).
139. Jafurulla, M., Aditya Kumar, G., Rao, B. D. & Chattopadhyay, A. A Critical Analysis of Molecular Mechanisms Underlying Membrane Cholesterol Sensitivity of GPCRs. Adv. Exp. Med. Biol. 1115, 21-52 (2019).
140. Gaibelet, G. et al. Cholesterol content drives distinct pharmacological behaviours of micro-opioid receptor in different microdomains of the CHO plasma membrane. Mol. Membr. Biol. 25, 423-435 (2008).
141. Levitt, E. S., Clark, M. J., Jenkins, P. M., Martens, J. R. & Traynor, J. R. Differential effect of membrane cholesterol removal on mu- and delta-opioid receptors: a parallel comparison of acute and chronic signaling to adenylyl cyclase. J. Biol. Chem. 284, 22108-22122 (2009).
142. Zheng, H. et al. Palmitoylation and membrane cholesterol stabilize μ-opioid receptor homodimerization and G protein coupling. BMC Cell Biol. 13, 6 (2012).
143. Michal, P., Rudajev, V., El-Fakahany, E. E. & Doležal, V. Membrane cholesterol content influences binding properties of muscarinic M2 receptors and differentially impacts activation of second messenger pathways. Eur. J. Pharmacol. 606, 50-60 (2009).
144. Bari, M., Paradisi, A., Pasquariello, N. & Maccarrone, M. Cholesterol-dependent modulation of type 1 cannabinoid receptors in nerve cells. J. Neurosci. Res. 81, 275-283 (2005).
145. Albert, A. D. & Boesze-Battaglia, K. The role of cholesterol in rod outer segment membranes. Prog. Lipid Res. 44, 99-124 (2005).
146. Paila, Y. D., Murty, M. R. V. S., Vairamani, M. & Chattopadhyay, A. Signaling by the human serotonin (1A) receptor is impaired in cellular model of Smith-Lemli-Opitz Syndrome. Biochim. Biophys. Acta 1778, 1508-1516 (2008).
147. Rodriguez, R. E. et al. Characterization of ZFOR1, a putative delta-opioid receptor from the teleost zebrafish (Danio rerio). Neurosci. Lett. 288, 207-210 (2000).
148. Sharpe, H. J., Stevens, T. J. & Munro, S. A comprehensive comparison of transmembrane domains reveals organelle-specific properties. Cell 142, 158-169 (2010).
149. Manglik, A. et al. Structure-based discovery of opioid analgesics with reduced side effects. Nature 537, 185-190 (2016).
150. Ehrlich, A. T. et al. Biased Signaling of the Mu Opioid Receptor Revealed in Native Neurons. iScience 14, 47-57 (2019).
151. Pyne, M. E. et al. A yeast platform for high-level synthesis of tetrahydroisoquinoline alkaloids. Nat. Commun. 11, 3337 (2020).
152. Ostrov, N. et al. A modular yeast biosensor for low-cost point-of-care pathogen detection. Sci. Adv. 3, e1603221 (2017).
153. Baker Brachmann, C. et al. Designer deletion strains derived from Saccharomyces cerevisiae S288C: A useful set of strains and plasmids for PCR-mediated gene disruption and other applications. Yeast 14, 115-132 (1998).
154. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015).
155. Bean, B. D. M., Whiteway, M. & Martin, V. J. J. The MyLO CRISPR-Cas9 toolkit: a markerless yeast localization and overexpression CRISPR-Cas9 toolkit. G3 GenesGenomesGenetics 12, jkac 154 (2022).
156. Gietz, R. D. & Schiestl, R. H. High-efficiency yeast transformation using the LiAc/SS carrier DNA/PEG method. Nat. Protoc. 2, 31-34 (2007).
157. Schindelin, J. et al. Fiji: an open-source platform for biological-image analysis. Nat. Methods 9, 676-682 (2012).
158. Lú Chau, T., Guillán, A., Roca, E., Núñez, M. J. & Lema, J. M. Population dynamics of a continuous fermentation of recombinant Saccharomyces cerevisiae using flow cytometry. Biotechnol. Prog. 17, 951-957 (2001).
159. Irving, A. J., McDonald, N. A. & Harkany, T. CB1 Cannabinoid Receptors: Molecular Biology, Second Messenger Coupling and Polarized Trafficking in Neurons. in Cannabinoids and the Brain (ed. Köfalvi, A.) 59-73 (Springer US, 2008). doi: 10.1007/978-O-387-74349-3_5.
160. Zou, S. & Kumar, U. Cannabinoid Receptors and the Endocannabinoid System: Signaling and Function in the Central Nervous System. Int. J. Mol. Sci. 19, 833 (2018).
161. Lu, H.-C. & Mackie, K. An introduction to the endogenous cannabinoid system. Biol. Psychiatry 79, 516-525 (2016).
162. Ranganathan, M. et al. Reduced Brain Cannabinoid Receptor Availability In Schizophrenia. Biol. Psychiatry 79, 997-1005 (2016).
163. Turcotte, C., Blanchet, M.-R., Laviolette, M. & Flamand, N. The CB2 receptor and its role as a regulator of inflammation. Cell. Mol. Life Sci. 73, 4449-4470 (2016).
164. Tai, S. & Fantegrossi, W. E. Synthetic Cannabinoids: Pharmacology, Behavioral Effects, and Abuse Potential. Curr. Addict. Rep. 1, 129-136 (2014).
165. Adams, A. J. et al. “Zombie” Outbreak Caused by the Synthetic Cannabinoid AMB-FUBINACA in New York. N. Engl. J. Med. 376, 235-242 (2017).
166. Lobato-Freitas, C. et al. Overview of Synthetic Cannabinoids ADB-FUBINACA and AMB-FUBINACA: Clinical, Analytical, and Forensic Implications. Pharmaceuticals 14, 186 (2021).
167. Tokarczyk, B., Jurczyk, A., Krupińska, J. & Adamowicz, P. Fatal intoxication with new synthetic cannabinoids 5F-MDMB-PICA and 4F-MDMB-BINACA-parent compounds and metabolite identification in blood, urine and cerebrospinal fluid. Forensic Sci. Med. Pathol. 1-10 (2022) doi: 10.1007/s12024-022-00492-3.
168. Hua, T. et al. Activation and Signaling Mechanism Revealed by Cannabinoid Receptor-Gi Complex Structures. Cell 180, 655-665.e18 (2020).
169. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015).
170. Andersson, H., D'Antona, A. M., Kendall, D. A., Heijne, G. V. & Chin, C.-N. Membrane Assembly of the Cannabinoid Receptor 1: Impact of a Long N-Terminal Tail. Mol. Pharmacol. 64, 570-577 (2003).
171. Hebert-Chatelain, E. et al. A cannabinoid link between mitochondria and memory. Nature 539, 555-559 (2016).
172. Smith, M. et al. The Effects of the Endocannabinoids Anandamide and 2-Arachidonoylglycerol on Human Osteoblast Proliferation and Differentiation. PLOS ONE 10, e0136546 (2015).
173. Miettinen, K. et al. A GPCR-based yeast biosensor for biomedical, biotechnological, and point-of-use cannabinoid determination. Nat. Commun. 13, 3664 (2022).
174. Gonsiorek, W. et al. Endocannabinoid 2-Arachidonyl Glycerol Is a Full Agonist through Human Type 2 Cannabinoid Receptor: Antagonism by Anandamide. Mol. Pharmacol. 57, 1045-1050 (2000).
175. Ishii, J. et al. Cell Wall Trapping of Autocrine Peptides for Human G-Protein-Coupled Receptors on the Yeast Cell Surface. PLOS ONE 7, e37136 (2012).
176. Bean, B. D. et al. Functional expression of opioid receptors and other human GPCRs in yeast engineered to produce human sterols. bioRxiv 2021.05.12.443385 (2021) doi: 10.1101/2021.05.12.443385.
177. Kapolka, N. J. et al. DCyFIR: a high-throughput CRISPR platform for multiplexed G protein-coupled receptor profiling and ligand discovery. Proc. Natl. Acad. Sci. 117, 13117-13126 (2020).
178. Gimpl, G. Interaction of G protein coupled receptors and cholesterol. Chem. Phys. Lipids 199, 61-73 (2016).
179. Sarkar, P. & Chattopadhyay, A. Cholesterol in GPCR Structures: Prevalence and Relevance. J. Membr. Biol. (2021) doi: 10.1007/s00232-021-00197-8.
180. Oddi, S. et al. Functional characterization of putative cholesterol binding sequence (CRAC) in human type-1 cannabinoid receptor. J. Neurochem. 116, 858-865 (2011).
181. Bari, M. et al. Effect of Lipid Rafts on Cb2 Receptor Signaling and 2-Arachidonoyl-Glycerol Metabolism in Human Immune Cells. J. Immunol. 177, 4971-4980 (2006).
182. Yeliseev, A. et al. Cholesterol as a modulator of cannabinoid receptor CB2 signaling. Sci. Rep. 11, 3706 (2021).
183. Scala, C. D., Fantini, J., Yahi, N., Barrantes, F. J. & Chahinian, H. Anandamide Revisited: How Cholesterol and Ceramides Control Receptor-Dependent and Receptor-Independent Signal Transmission Pathways of a Lipid Neurotransmitter. Biomolecules 8, 31 (2018).
184. LoVerme, J. et al. Synthesis and characterization of a peripherally restricted CB1 cannabinoid antagonist, URB447, that reduces feeding and body-weight gain in mice. Bioorg. Med. Chem. Lett. 19, 639-643 (2009).
185. Eissenstat, M. A. et al. Aminoalkylindoles: Structure-Activity Relationships of Novel Cannabinoid Mimetics. J. Med. Chem. 38, 3094-3105 (1995).
186. Jüttler, E. et al. The cannabinoid dexanabinol is an inhibitor of the nuclear factor-kappa B (NF-κB). Neuropharmacology 47, 580-592 (2004).
187. Mechoulam, R., Ben-Zvi, Z. & Gaoni, Y. Hashish-XIII: On the nature of the beam test. Tetrahedron 24, 5615-5624 (1968).
188. Bb, Y. et al. In vitro and in vivo characterization of A-796260: a selective cannabinoid CB2 receptor agonist exhibiting analgesic activity in rodent pain models. Br. J. Pharmacol. 153, (2008).
189. Yao, B. B. et al. In vitro pharmacological characterization of AM1241: a protean agonist at the cannabinoid CB2 receptor? Br. J. Pharmacol. 149, 145 (2006).
190. Diaz, P. et al. 6-Methoxy-N-alkyl isatin acylhydrazone derivatives as a novel series of potent selective cannabinoid receptor 2 inverse agonists: design, synthesis, and binding mode prediction. J. Med. Chem. 52, 433-444 (2009).
191. Frost, J. M. et al. Indol-3-ylcycloalkyl Ketones: Effects of N1 Substituted Indole Side Chain Variations on CB2 Cannabinoid Receptor Activity. J. Med. Chem. 53, 295-315 (2010).
192. Russo, E. B. Taming THC: potential cannabis synergy and phytocannabinoid-terpenoid entourage effects. Br. J. Pharmacol. 163, 1344-1364 (2011).
193. Fletcher, R. S. & Mckay, J. U.S. patent Ser. No. 10/499,584-Industrial hemp Cannabis cultivars and seeds with stable cannabinoid profiles. (2019).
194. Fischedick J, E. S. Cannabinoids and Terpenes as Chemotaxonomic Markers in Cannabis. Nat. Prod. Chem. Res. 03, (2015).
195. Sharp, H. et al. Totarol, totaradiol and ferruginol: three diterpenes from Thuja plicata (Cupressaceae). Biochem. Syst. Ecol. 29, 215-217 (2001).
196. Gao, Y. et al. Totarol prevents neuronal injury in vitro and ameliorates brain ischemic stroke: Potential roles of Akt activation and HO-1 induction. Toxicol. Appl. Pharmacol. 289, 142-154 (2015).
197. Kubo, I., Muroi, H. & Himejima, M. Antibacterial Activity of Totarol and Its Potentiation. J. Nat. Prod. 55, 1436-1440 (1992).
198. Cannaert, A. et al. Synthesis and in Vitro Cannabinoid Receptor 1 Activity of Recently Detected Synthetic Cannabinoids 4F-MDMB-BICA, 5F-MPP-PICA, MMB-4en-PICA, CUMYL-CBMICA, ADB-BINACA, APP-BINACA, 4F-MDMB-BINACA, MDMB-4en-PINACA, A-CHMINACA, 5F-AB-P7AICA, 5F-MDMB-P7AICA, and 5F-AP7AICA. ACS Chem. Neurosci. 11, 4434-4446 (2020).
199. Lie, W. et al. Diagnosing intake and rationalizing toxicities associated with 5F-MDMB-PINACA and 4F-MDMB-BINACA abuse. Arch. Toxicol. 95, 489-508 (2021).
200. Banister, S. D. et al. Pharmacology of Indole and Indazole Synthetic Cannabinoid Designer Drugs AB-FUBINACA, ADB-FUBINACA, AB-PINACA, ADB-PINACA, 5F-AB-PINACA, 5F-ADB-PINACA, ADBICA, and 5F-ADBICA. ACS Chem. Neurosci. 6, 1546-1559 (2015).
201. Kumar, K. K. et al. Structure of a Signaling Cannabinoid Receptor 1-G Protein Complex. Cell 176, 448-458.e12 (2019).
202. Armenian, P. et al. Intoxication from the novel synthetic cannabinoids AB-PINACA and ADB-PINACA: A case series and review of the literature. Neuropharmacology 134, 82-91 (2018).
203. Xing, C. et al. Cryo-EM Structure of the Human Cannabinoid Receptor CB2-Gi Signaling Complex. Cell 180, 645-654.e13 (2020).
204. Luo, X. et al. Complete biosynthesis of cannabinoids and their unnatural analogues in yeast. Nature 567, 123-126 (2019).
205. Ehrenworth, A. M., Claiborne, T. & Peralta-Yahya, P. Medium-Throughput Screen of Microbially Produced Serotonin via a G-Protein-Coupled Receptor-Based Sensor. Biochemistry 56, 5471-5475 (2017).
206. Mukherjee, K., Bhattacharyya, S. & Peralta-Yahya, P. GPCR-Based Chemical Biosensors for Medium-Chain Fatty Acids. ACS Synth. Biol. 4, 1261-1269 (2015).
207. Chin, C.-L. et al. Differential effects of cannabinoid receptor agonists on regional brain activity using pharmacological MRI. Br. J. Pharmacol. 153, 367-379 (2008).
208. Hillard, C. J. et al. Synthesis and Characterization of Potent and Selective Agonists of the Neuronal Cannabinoid Receptor (CB1). J. Pharmacol. Exp. Ther. 289, 1427-1433 (1999).
209. Yao, B. B. et al. Characterization of a Cannabinoid CB2 Receptor-Selective Agonist, A-836339 [2,2,3,3-Tetramethyl-cyclopropanecarboxylic Acid [3-(2-Methoxy-ethyl)-4,5-dimethyl-3H-thiazol-(2Z)-ylidene]-amide], Using in Vitro Pharmacological Assays, in Vivo Pain Models, and Pharmacological Magnetic Resonance Imaging. J. Pharmacol. Exp. Ther. 328, 141-151 (2009).
210. Ben-Shabat, S. et al. An entourage effect: inactive endogenous fatty acid glycerol esters enhance 2-arachidonoyl-glycerol cannabinoid activity. Eur. J. Pharmacol. 353, 23-31 (1998).
211. Mechoulam, R. et al. Identification of an endogenous 2-monoglyceride, present in canine gut, that binds to cannabinoid receptors. Biochem. Pharmacol. 50, 83-90 (1995).
212. Adasme, M. F. et al. PLIP 2021: expanding the scope of the protein-ligand interaction profiler to DNA and RNA. Nucleic Acids Res. 49, W530-W534 (2021).
213. Benyamin, R. et al. Opioid Complications and Side Effects. Pain Physician 11, S105-20 (2008).
214. Porro, D., Sauer, M., Branduardi, P. & Mattanovich, D. Recombinant protein production in yeasts. Mol. Biotechnol. 31, 245-259 (2005).
215. Davenport, A. P., Scully, C. C. G., de Graaf, C., Brown, A. J. H. & Maguire, J. J. Advances in therapeutic peptides targeting G protein-coupled receptors. Nat. Rev. Drug Discov. 19, 389-413 (2020).
216. Cherf, G. M. & Cochran, J. R. Applications of yeast surface display for protein engineering. Methods Mol. Biol. Clifton NJ 1319, 155-175 (2015).
217. Goodnow, R. A., Dumelin, C. E. & Keefe, A. D. DNA-encoded chemistry: enabling the deeper sampling of chemical space. Nat. Rev. Drug Discov. 16, 131-147 (2017).
218. Muttenthaler, M., King, G. F., Adams, D. J. & Alewood, P. F. Trends in peptide drug discovery. Nat. Rev. Drug Discov. 20, 309-325 (2021).
219. Pathan, H. & Williams, J. Basic opioid pharmacology: an update. Br. J. Pain 6, 11-16 (2012).
220. Quirion, B., Bergeron, F., Blais, V. & Gendron, L. The Delta-Opioid Receptor; a Target for the Treatment of Pain. Front. Mol. Neurosci. 13, (2020).
221. Viscusi, E. R. et al. A randomized, phase 2 study investigating TRV130, a biased ligand of the μ-opioid receptor, for the intravenous treatment of acute pain. Pain 157, 264-272 (2016).
222. Valentino, R. J. & Volkow, N. D. Untangling the complexity of opioid receptor function. Neuropsychopharmacology 43, 2514-2520 (2018).
223. O'Connor, C. et al. NMR structure and dynamics of the agonist dynorphin peptide bound to the human kappa opioid receptor. Proc. Natl. Acad. Sci. 112, 11852-11857 (2015).
224. Che, T. et al. Structure of the Nanobody-Stabilized Active State of the Kappa Opioid Receptor. Cell 172, 55-67.e15 (2018).
225. Bruchas, M. R., Land, B. B. & Chavkin, C. The dynorphin/kappa opioid system as a modulator of stress-induced and pro-addictive behaviors. Brain Res. 1314, 44-55 (2010).
226. Bruchas, M. R. & Roth, B. L. New Technologies for Elucidating Opioid Receptor Function. Trends Pharmacol. Sci. 37, 279-289 (2016).
227. Manchikanti, L. et al. Opioid Epidemic in the United States. Pain Physician 15, ES9-ES38 (2012).
228. Fricker, L. D., Margolis, E. B., Gomes, I. & Devi, L. A. Five Decades of Research on Opioid Peptides: Current Knowledge and Unanswered Questions. Mol. Pharmacol. 98, 96-108 (2020).
229. Granier, S. et al. Structure of the 8-opioid receptor bound to naltrindole. Nature 485, 400-404 (2012).
230. Portoghese, P. S. Bivalent ligands and the message-address concept in the design of selective opioid receptor antagonists. Trends Pharmacol. Sci. 10, 230-235 (1989).
231. Cheng, J. et al. Computational insights into the subtype selectivity and “message-address-efficacy” mechanisms of opioid receptors through JDTic binding and unbinding. Acta Pharmacol. Sin. 39, 482-491 (2018).
232. Lai, J. et al. Dynorphin A activates bradykinin receptors to maintain neuropathic pain. Nat. Neurosci. 9, 1534-1540 (2006).
233. Lung, F.-D. T. et al. Effects of Modifications of Residues in Position 3 of Dynorphin A (1-11)-NH2 on κ Receptor Selectivity and Potency. J. Med. Chem. 39, 2456-2460 (1996).
234. Vardy, E. et al. Chemotype-selective Modes of Action of κ-Opioid Receptor Agonists. J. Biol. Chem. 288, 34470-34483 (2013).
235. Kimura, S. et al. Probable precursors of [Leu]enkephalin and [Met]enkephalin in adrenal medulla: peptides of 3-5 kilodaltons. Proc. Natl. Acad. Sci. 77, 1681-1685 (1980).
236. Bloom, F. E. The Endorphins: A Growing Family of Pharmacologically Pertinent Peptides. Annu. Rev. Pharmacol. Toxicol. 23, 151-170 (1983).
237. SCHWARZER, C. 30 Years of Dynorphins-New Insights on Their Functions in Neuropsychiatric Diseases. Pharmacol. Ther. 123, 353-370 (2009).
238. Sarkar, P. & Chattopadhyay, A. Cholesterol in GPCR Structures: Prevalence and Relevance. J. Membr. Biol. 255, 99-106 (2022).
239. Parks, L. W. & Casey, W. M. Physiological implications of sterol biosynthesis in yeast. Annu. Rev. Microbiol. 49, 95-117 (1995).
240. Yoshimoto, N. et al. High-throughput de novo screening of receptor agonists with an automated single-cell analysis and isolation system. Sci. Rep. 4, 4242 (2014).
241. Nakamura, Y., Ishii, J. & Kondo, A. Bright fluorescence monitoring system utilizing Zoanthus sp. green fluorescent protein (ZsGreen) for human G-protein-coupled receptor signaling in microbial yeast cells. PloS One 8, e82237 (2013).
242. Sato, N. et al. Long anchor using Flo1 protein enhances reactivity of cell surface-displayed glucoamylase to polymer substrates. Appl. Microbiol. Biotechnol. 60, 469-474 (2002).
243. Nakamura, Y. et al. Biosensing Techniques in Yeast: G-Protein Signaling and Protein-Protein Interaction Assays for Monitoring Ligand Stimulation and Oligomer Formation of Heterologous GPCRs. Peripheral Membrane Proteins (IntechOpen, 2018). doi: 10.5772/intechopen.76330.
244. Chavkin, C. & Goldstein, A. Specific receptor for the opioid peptide dynorphin: structure—activity relationships. Proc. Natl. Acad. Sci. U.S.A 78, 6543-6547 (1981).
245. Metzger, T. G., Paterlini, M. G., Ferguson, D. M. & Portoghese, P. S. Investigation of the Selectivity of Oxymorphone- and Naltrexone-Derived Ligands via Site-Directed Mutagenesis of Opioid Receptors: Exploring the ‘Address’ Recognition Locus. J. Med. Chem. 44, 857-862 (2001).
246. Xue, J. C. et al. Differential binding domains of peptide and non-peptide ligands in the cloned rat kappa opioid receptor. J. Biol. Chem. 269, 30195-30199 (1994).
247. Marie-Pepin, C., Yue, S. Y., Roberts, E., Wahlestedt, C. & Walker, P. Novel “Restoration of Function” Mutagenesis Strategy to Identify Amino Acids of the 8-Opioid Receptor Involved in Ligand Binding*. J. Biol. Chem. 272, 9260-9267 (1997).
248. Bass, R. T. et al. Identification and characterization of novel somatostatin antagonists. Mol. Pharmacol. 50, 709-715 (1996).
249. Campbell, R. M. et al. Selective A1-adenosine receptor antagonists identified using yeast Saccharomyces cerevisiae functional assays. Bioorg. Med. Chem. Lett. 9, 2413-2418 (1999).
250. Fenalti, G. et al. Molecular control of 8-opioid receptor signalling. Nature 506, 191-196 (2014).
251. Metzger, T. G. & Ferguson, D. M. On the role of extracellular loops of opioid receptors in conferring ligand selectivity. FEBS Lett. 375, 1-4 (1995).
252. Li, H. & Durbin, R. Fast and accurate short read alignment with Burrows-Wheeler transform. Bioinformatics 25, 1754-1760 (2009).
253. Danecek, P. et al. Twelve years of SAMtools and BCFtools. GigaScience 10, giab008 (2021).
254. R: a language and environment for statistical computing. https://www.gbif.org/tool/81287/r-a-language-and-environment-for-statistical-computing.
255. Synthetic repertoires derived from convalescent COVID-19 patients enable discovery of SARS-COV-2 neutralizing antibodies and a novel quaternary binding modality|bioRxiv. https://www.biorxiv.org/content/10.1101/2021.04.07.438849v2.full.
256. de Ruijter, J. C., Koskela, E. V. & Frey, A. D. Enhancing antibody folding and secretion by tailoring the Saccharomyces cerevisiae endoplasmic reticulum. Microb. Cell Factories 15, 87 (2016).
257. Shusta, E. V., Raines, R. T., Pluckthun, A. & Wittrup, K. D. Increasing the secretory capacity of Saccharomyces cerevisiae for production of single-chain antibody fragments. Nat. Biotechnol. 16, 773-777 (1998).
258. Bortolato, A. et al. Structure of Class B GPCRs: new horizons for drug discovery. Br. J. Pharmacol. 171, 3132-3145 (2014).
259. Chun, L., Zhang, W. & Liu, J. Structure and ligand recognition of class C GPCRs. Acta Pharmacol. Sin. 33, 312-323 (2012).
260. Miettinen, K. et al. A GPCR-based yeast biosensor for biomedical, biotechnological, and point-of-use cannabinoid determination. Nat. Commun. 13, 3664 (2022).
261. Gurevich, V. V. & Gurevich, E. V. GPCR Signaling Regulation: The Role of GRKs and Arrestins. Front. Pharmacol. 10, (2019).
262. Fowler, D. M. & Fields, S. Deep mutational scanning: a new style of protein science. Nat. Methods 11, 801-807 (2014).
263. Amorosi, C. J. et al. Massively parallel characterization of CYP2C9 variant enzyme activity and abundance. Am. J. Hum. Genet. 108, 1735-1751 (2021).
264. Sun, S. et al. A proactive genotype-to-patient-phenotype map for cystathionine beta-synthase. Genome Med. 12, 13 (2020).
265. Weile, J. et al. A framework for exhaustively mapping functional missense variants. Mol. Syst. Biol. 13, 957 (2017).
266. Di Roberto, R. B., Chang, B. & Peisajovich, S. G. The directed evolution of ligand specificity in a GPCR and the unequal contributions of efficacy and affinity. Sci. Rep. 7, 16012 (2017).

### Tables

TABLE 1
Top-performing motifs unique for individual
receptors identified for further analysis.

| Receptor | Library | Motif | Metric |
|---|---|---|---|
| DOR | 1 | YGFD | positive count |
| DOR | 1 | YGME | positive count |
| DOR | 1 | YGLD | positive count |
| DOR | 1 | YGMD | positive count |
| DOR | 1 | WWPD | positive count |
| DOR | 1 | YGFG | positive count |
| KOR | 1 | SVPF | positive count |
| KOR | 1 | CDIQ | positive count |
| KOR | 1 | SHYV | positive count |
| KOR | 1 | SAWP | positive count |
| KOR | 1 | SRVF | positive count |
| KOR | 1 | YQRF | positive count |
| MOR | 1 | GLVN | positive count |
| MOR | 1 | LSPI | positive count |
| MOR | 1 | LVLL | positive count |
| DOR | 2 | GSGR | positive count |
| DOR | 2 | SSSG | positive count |
| DOR | 2 | GGSG | positive count |
| DOR | 2 | SSRG | positive count |
| DOR | 2 | SSGL | positive count |
| KOR | 2 | YSYA | positive count |
| KOR | 2 | YRLR | positive count |
| KOR | 2 | YRLA | positive count |
| KOR | 2 | YSRW | positive count |
| MOR | 2 | MGSV | positive count |
| MOR | 2 | MVAT | positive count |
| MOR | 2 | RCCA | positive count |
| MOR | 2 | MNYP | positive count |
| DOR | 1 | LSLL | log proportion |
| KOR | 1 | LLLL | log proportion |
| MOR | 1 | LLSL | log proportion |
| DOR | 2 | GSSS | log proportion |
| KOR | 2 | LRRR | log proportion |
| MOR | 2 | MRRR | log proportion |
| DOR | 1 | YGPL | log proportion w/count filter |
| KOR | 1 | SLRF | log proportion w/count filter |
| MOR | 1 | LSSL | log proportion w/count filter |
| KOR | 2 | YRRR | log proportion w/count filter |
| MOR | 2 | MSRR | log proportion w/count filter |

TABLE 2
Coverage results of the untransformed dynorphin libraries 1 and 2.

| dynLib | 1 | 2 |
|---|---|---|
| Analysis version | 2 | 2 |
| Theoretical NT complexity | 1048576 | 1048576 |
| Total NT seqs | 1932166 | 641339 |
| Unique NT seqs | 458121 | 451341 |
| Theoretical AA complexity w/stops | 194481 | 194481 |
| Theoretical AA complexity no/stops | 160000 | 160000 |
| Unique AA seqs | 142872 | 162126 |
| Good AA seqs (no stop codon) | 117803 | 134917 |
| Bad AA seqs (have stop codon) | 25069 | 27209 |
| Percentage of AA complexity no/stops | 73.46 | 83.36 |

TABLE 3
Genes used in Example 1

| Gene | | |
|---|---|---|
| Name | | |
| (alias) | Type | Sequence |
| HsMOR | opioid | ATGGATTCTTCAGCAGCTCCAACAAATGCCTCTAACTGC |
| | receptor | ACTGATGCTTTGGCTTATTCTTCTTGTTCTCCAGCTCCAT |
| | | CTCCAGGTTCTTGGGTTAATTTGTCTCATTTGGATGGTAA |
| | | CTTGTCTGATCCATGTGGTCCAAATAGAACTGACTTAGG |
| | | TGGTAGAGATTCTTTGTGTCCACCAACTGGTTCTCCATCT |
| | | ATGATTACTGCTATTACCATTATGGCCCTGTACTCTATCG |
| | | TTTGTGTTGTTGGTTTGTTCGGTAACTTCTTGGTTATGTA |
| | | CGTTATCGTCAGATACACCAAGATGAAGACTGCTACCA |
| | | ACATCTACATTTTCAACTTGGCTTTGGCTGATGCATTGG |
| | | CAACTTCTACTTTGCCATTTCAATCCGTCAATTACTTGAT |
| | | GGGTACTTGGCCATTTGGTACTATTTTGTGCAAGATCGT |
| | | TATCTCCATCGACTACTACAACATGTTCACCTCTATTTTC |
| | | ACCTTGTGCACCATGTCCGTTGATAGATATATTGCTGTTT |
| | | GCCATCCAGTTAAGGCCTTGGATTTTAGAACTCCAAGAA |
| | | ACGCCAAGATTATCAACGTCTGCAATTGGATTTTGTCCT |
| | | CCGCTATTGGTTTGCCAGTTATGTTTATGGCTACTACCA |
| | | AGTACAGACAAGGTTCTATTGATTGCACTTTGACCTTCT |
| | | CTCATCCAACTTGGTATTGGGAAAACTTGTTGAAGATCT |
| | | GCGTTTTCATTTTCGCCTTCATCATGCCAGTTTTGATTAT |
| | | CACTGTTTGCTACGGCTTGATGATCTTGAGATTGAAGTC |
| | | TGTCAGAATGCTGTCCGGTTCTAAAGAAAAGGATAGAA |
| | | ACTTGAGAAGGATCACCAGAATGGTTTTGGTTGTTGTTG |
| | | CTGTTTTCATCGTTTGTTGGACCCCAATTCATATCTACGT |
| | | TATCATCAAGGCTTTGGTCACTATTCCAGAAACTACTTT |
| | | CCAAACTGTTTCCTGGCATTTCTGTATTGCTTTGGGTTAC |
| | | ACTAACTCTTGTTTGAACCCAGTCTTGTACGCTTTTTTGG |
| | | ACGAAAACTTCAAGAGATGCTTCCGTGAATTCTGTATTC |
| | | CAACCTCCTCTAACATCGAACAACAAAACTCTACCAGA |
| | | ATCAGACAGAACACTAGAGATCATCCATCTACTGCTAAC |
| | | ACTGTTGATAGAACTAACCACCAATTGGAGAACTTGGA |
| | | AGCTGAAACTGCTCCATTGCCA (SEQ ID NO: 1) |
| HsKOR | opioid | ATGGAACCAGCTCCTTCTGCAGGTGCTGAATTGCAGCCA |
| | receptor | CCTTTGTTTGCTAATGCTTCTGATGCTTATCCATCTGCTT |
| | | TTCCATCAGCTGGTGCTAATGCATCAGGTCCACCAGGTG |
| | | CTAGATCTGCTTCTTCTTTGGCTTTGGCTATTGCAATTAC |
| | | TGCCTTGTATTCTGCTGTTTGTGCTGTTGGTTTGTTGGGT |
| | | AATGTTTTGGTTATGTTCGGTATCGTCAGATACACTAAG |
| | | ATGAAGACTGCTACCAACATCTACATTTTCAATTTGGCA |
| | | TTGGCTGATGCTTTGGCCACTTCTACTTTGCCATTTCAAT |
| | | CTGCTAAGTACTTGATGGAAACTTGGCCATTCGGTGAAT |
| | | TATTGTGTAAGGCCGTTTTGTCCATCGACTACTACAATA |
| | | TGTTCACCTCCATTTTCACCCTGACCATGATGTCTGTTGA |
| | | TAGGTATATTGCAGTTTGCCATCCAGTTAAGGCTTTGGA |
| | | TTTTAGAACTCCAGCTAAGGCCAAGTTGATTAACATCTG |
| | | TATTTGGGTTTTAGCCTCCGGTGTTGGTGTTCCAATTATG |
| | | GTTATGGCTGTTACTAGACCAAGAGATGGTGCTGTTGTA |
| | | TGTATGTTGCAATTTCCTTCTCCATCTTGGTACTGGGATA |
| | | CTGTTACTAAGATTTGCGTTTTCTTGTTCGCCTTCGTTGT |
| | | TCCAATCTTGATTATTACCGTTTGCTACGGCTTGATGTTG |
| | | TTGAGATTGAGATCCGTCAGATTGCTGTCTGGTTCTAAA |
| | | GAAAAGGACAGATCCTTGAGAAGAATCACCAGAATGGT |
| | | TTTGGTTGTTGTTGGTGCTTTCGTTGTTTGCTGGGCTCCA |
| | | ATTCATATTTTCGTTATCGTTTGGACCTTGGTCGACATCG |
| | | ATAGAAGAGATCCATTAGTTGTTGCTGCCTTGCATTTGT |
| | | GTATTGCTTTGGGTTATGCTAACTCCTCATTGAATCCAGT |
| | | CTTGTACGCTTTTTTGGACGAAAACTTCAAGAGATGCTT |
| | | CAGACAATTGTGTAGAAAGCCATGTGGTAGACCAGATC |
| | | CATCTTCATTTTCTAGAGCTAGAGAAGCTACTGCCAGAG |
| | | AAAGAGTTACTGCTTGTACTCCATCTGATGGTCCAGGTG |
| | | GTGGTGCTGCAGCT (SEQ ID NO: 2) |
| HsDOR | opioid | ATGGATTCTCCAATTCAAATTTTCAGAGGTGAACCAGGT |
| | receptor | CCAACTTGTGCTCCATCTGCTTGTTTGCCACCAAATTCTT |
| | | CAGCTTGGTTTCCAGGTTGGGCTGAACCAGATTCTAATG |
| | | GTTCTGCTGGTTCTGAAGATGCTCAATTGGAACCAGCTC |
| | | ATATTTCTCCAGCTATTCCAGTTATTATCACCGCCGTTTA |
| | | CTCTGTTGTTTTCGTTGTTGGTTTGGTCGGTAACTCTTTG |
| | | GTTATGTTCGTTATCATCAGGTACACCAAGATGAAGACT |
| | | GCTACCAACATCTACATTTTCAACTTGGCTTTGGCTGAT |
| | | GCTTTGGTTACTACTACTATGCCATTCCAATCTACCGTCT |
| | | ACTTGATGAATTCTTGGCCATTTGGTGATGTCTTGTGCA |
| | | AGATCGTTATCTCCATTGACTACTACAACATGTTCACCT |
| | | CCATTTTCACTCTGACCATGATGTCCGTTGATAGGTATA |
| | | TTGCTGTTTGCCATCCAGTTAAGGCTTTGGATTTTAGAA |
| | | CTCCATTGAAGGCCAAGATCATCAACATCTGTATTTGGC |
| | | TGTTGTCCTCCTCTGTTGGTATTTCTGCTATAGTTTTAGG |
| | | TGGCACCAAGGTTAGAGAAGATGTTGACGTTATTGAAT |
| | | GCTCCTTGCAATTCCCAGATGATGATTATTCTTGGTGGG |
| | | ACTTGTTCATGAAGATCTGCGTTTTCATTTTCGCCTTCGT |
| | | TATCCCAGTTTTGATCATCATCGTTTGCTACACCTTGATG |
| | | ATCCTGAGATTGAAATCTGTCAGACTGTTGTCTGGTTCC |
| | | AGAGAAAAGGATAGAAACTTGAGAAGAATCACCAGATT |
| | | GGTTTTGGTTGTTGTTGCTGTTTTTGTTGTTTGCTGGACC |
| | | CCAATTCACATCTTCATTTTGGTTGAAGCTTTGGGTTCTA |
| | | CCTCTCATTCTACTGCTGCTTTGTCATCTTACTATTTCTG |
| | | CATTGCCTTGGGTTACACCAACTCTTCATTGAATCCAAT |
| | | CTTGTACGCTTTCTTGGACGAAAACTTCAAGAGATGCTT |
| | | CAGAGATTTCTGCTTCCCATTGAAGATGAGAATGGAAA |
| | | GACAATCCACCTCCAGAGTTAGAAACACTGTTCAAGATC |
| | | CAGCTTACTTGAGAGATATCGATGGTATGAACAAGCCA |
| | | GTC (SEQ ID NO: 3) |
| Flo42 | tethering | AGCAGCATGGTAGGATATAGTACAGCTTCTTTAGAAAT |
| | component | TTCAACGTATGCTGGCAGTGCCAACAGCTTACTGGCCG |
| | | GTAGTGGTTTAAGTGTCTTCATTGCGTCCTTATTGCTGG |
| | | CAATTATTTAA (SEQ ID NO: 4) |
| dynorphin | peptide | TATGGGGGTTTCCTGAGGCGTATA (SEQ ID NO: 5) |
| A 1-8 | | |
| dynorphin | peptide | TATGGGGGTTTCCTGAGGCGTATAAGGCCGAAGTTAAA |
| A 1-13 | | G (SEQ ID NO: 6) |
| MF alpha | tethering | ATGAGATTTCCTTCAATTTTTACTGCAGTTTTATTCGCA |
| leader | component | GCATCCTCCGCATTAGCTGCTCCAGTCAACACTACAAC |
| | | AGAAGATGAAACGGCACAAATTCCGGCTGAAGCTGTCA |
| | | TCGGTTACTTAGATTTAGAAGGGGATTTCGATGTTGCTG |
| | | TTTTGCCATTTTCCAACAGCACAAATAACGGGTTATTGT |
| | | TTATAAATACTACTATTGCCAGCATTGCTGCTAAAGAA |
| | | GAAGGAGTCTCTTTGGATAAAAGA (SEQ ID NO: 7) |
| MF alpha | peptide | TGGCATTGGTTGCAATTGAAACCAGGTCAACCAATGTA |
| peptide | control | T (SEQ ID NO: 8) |
| glycine- | tethering | GGCGGTGGAAGCGGTGGTGGAGGGAGCGGTGGGGGTT |
| serine | component | CTGGGGGCGGAGGGAGT (SEQ ID NO: 9) |
| linker | | |

TABLE 4
Strains used in Example 1

| Strain | Description | Genotype |
|---|---|---|
| JG05- | GPCR signaling | BY4741 fig1Δ::ZsGreen1 gal2Δ sst2Δ |
| 2.0 | strain (Gαi3 | ste2Δ far1Δ GPA1(468-472Δ)- |
| | G-alpha) | GNAI3(350-354) |
| CJM263 | GPCR signaling | BY4741 fig1Δ::ZsGreen1 gal2Δ sst2Δ |
| | strain (Gαi3 | ste2Δ far1Δ GPA1(468-472Δ)- |
| | G-alpha) | GNAI3(350-354) erg5Δ::TDH3pr- |
| | | Dr.DHCR7 erg6Δ::CCW12pr-Dr.DHCR24 |

TABLE 5
Plasmids Used in Example 1

| Plasmid | | | Yeast Marker |
|---|---|---|---|
| name | Description | Resistance | and Expression |
| AAR1 GFP | DO vector for tethered peptide libraries, | Kan | HIS3 IV |
| DO HO′ | peptide and linker replace GFP | | |
| 302 | pHHF2/dynorphin A 1-8 tethered/GS linker | Kan | Cen6 HIS3 |
| 303 | pGal/dynorphin A 1-8 tethered/GS linker | Kan | HIS3 IV |
| 304 | pGal/MFalpha tethered/GS linker | Kan | HIS3 IV |
| 305 | pGal/tethered GS linker | Kan | HIS3 IV |
| 306 | pHHF2/dynorphin A 1-13 tethered/GS linker | Kan | HIS3 IV |
| 307 | pHHF2/dynorphin A 1-8 tethered N-term/GS linker | Kan | Cen6 HIS3 |
| 308 | pHHF2/dynorphin A 1-13 tethered N-term/GS linker | Kan | Cen6 HIS3 |
| 225 | pCCW12-HsMOR-SSA1 terminator-2u URA backbone | Kan | 2u URA |
| 226 | pCCW12-HsKOR-SSA1 terminator-2u URA backbone | Kan | 2u URA |
| 227 | pCCW12-HsDOR-SSA1 terminator-2u URA backbone | Kan | 2u URA |
| 228 | pTDH3-HsMOR-SSA1 terminator-URA IV backbone | Kan | URA IV |
| 229 | pTDH3-HsKOR-SSA1 terminator-URA IV backbone | Kan | URA IV |
| 230 | pTDH3-HsDOR-SSA1 terminator-URA IV backbone | Kan | URA IV |

TABLE 6
Opioid Receptor Peptides
HsDOR-specific:
ADMSLRRI (SEQ ID NO: 10)
WWPDLRRI (SEQ ID NO: 11)
YGFDLRRI (SEQ ID NO: 12)
YGMELRRI (SEQ ID NO: 13)
YGGFGGSG (SEQ ID NO: 14)
YGGFGSGR (SEQ ID NO: 15)
YGGFSSGL (SEQ ID NO: 16)
YGGFSSRG (SEQ ID NO: 17)
YGGFSSSG (SEQ ID NO: 18)
HsKOR-specific:
CDIQLRRI (SEQ ID NO: 19)
SAWPLRRI (SEQ ID NO: 20)
SHYVLRRI (SEQ ID NO: 21)
SVPFLRRI (SEQ ID NO: 22)
YQRFLRRI (SEQ ID NO: 23)
YFFGYRLA (SEQ ID NO: 24)
YGGFYRLR (SEQ ID NO: 25)
YGGFYSRW (SEQ ID NO: 26)
YGGFYSYA (SEQ ID NO: 27)
HsMOR-specific:
GLVNLRRI (SEQ ID NO: 28)
LVLLLRRI (SEQ ID NO: 29)
YGGFMGSV (SEQ ID NO: 30)
YGGFMNYP (SEQ ID NO: 31)
YGGFMVAT (SEQ ID NO: 32)
YGGFRCCA (SEQ ID NO: 33)

1. A non-naturally occurring test molecule for use in a cell-based screening method, wherein the molecule comprises:
a) a molecule of interest comprising a message/address peptide;
b) a linker sequence; and
c) an anchor sequence which anchors the test molecule to a plasma membrane of the cell.

2. The non-naturally occurring test molecule of claim 1, wherein the molecule of interest is selected from the group comprising a polypeptide, a peptide, a small molecule, a natural product, a peptidomimetic, a nucleic acid, a lipid, lipopeptide, or a carbohydrate.

3. The non-naturally occurring test molecule of claim 2, wherein the molecule of interest is a protein.

4. The non-naturally occurring test molecule of claim 3, wherein the test molecule, which is a protein, further comprises a leader sequence which targets the test molecule for cell secretion.

5. The non-naturally occurring test molecule of claim 4, wherein the leader sequence comprises MF alpha leader sequence.

6. The non-naturally occurring test molecule of claim 1, wherein the message/address peptide originates from, or is a derivative of, at least one of the enkephalin, dynorphin and endorphin peptides.

7. The non-naturally occurring test molecule of claim 6, wherein the message/address peptide is eight amino acids in length.

8. The non-naturally occurring test molecule of claim 7, wherein the message portion of the message/address peptide comprises the amino acids tyrosine-glycine-glycine-phenylalanine.

9. The non-naturally occurring test molecule of claim 6, wherein the address portion of the message/address peptide is four amino acids in length but varies in composition.

10. The non-naturally occurring test molecule of claim 1, wherein the message/address peptide comprises any of SEQ ID NOS: 10-33.

11. The non-naturally occurring test molecule of claim 1, wherein the linker sequence is 12-24 amino acids in length.

12. The non-naturally occurring test molecule of claim 1, wherein the linker sequence consists of glycine and serine residues.

13. The non-naturally occurring test molecule of claim 12, wherein the linker sequence comprises SEQ ID NO: 34.

14. The non-naturally occurring test molecule of claim 1, wherein the anchor sequence comprises Flo1 or a derivative thereof.

15. The non-naturally occurring test molecule of claim 14, wherein the anchor sequence comprises Flo42.

16. The non-naturally occurring test molecule of claim 1, wherein the test molecule is in a eukaryotic host cell.

17. (canceled)

18. (canceled)

19. The non-naturally occurring test molecule of claim 16, wherein the test molecule is not native to the eukaryotic host cell.

20. A method of screening to determine interaction between a capture protein and a molecule of interest, the method comprising the steps of:
a) providing the molecule of claim 1;
b) providing a eukaryotic cell, wherein said eukaryotic cell comprises a non-endogenous capture protein;
c) allowing the molecule of interest and the non-endogenous capture protein to interact;
d) determining interaction between the molecule of interest and the capture protein.

21-45. (canceled)

46. A eukaryotic cell for use in a high throughput screening assay, wherein said cell comprises:
a) One or more test molecules, wherein said test molecule comprises the non-naturally occurring molecule of claim 1, test molecule is anchored to the cell's plasma membrane;
b) A capture protein, wherein said capture protein is found in the cell's plasma membrane, and is within proximity to the molecule of interest such that the molecule of interest and the capture protein can interact.
c) A reporter function that is activated upon binding between the test molecule and the capture protein.

47-53. (canceled)

54. An expression vector encoding a protein of interest, wherein said protein of interest comprises a) a leader sequence which targets the protein for cell secretion; b) a molecule of interest coupled to a message/address peptide; c) a linker sequence; and d) an anchor sequence which anchors the molecule to a plasma membrane of the cell.

55-56. (canceled)
